(19) United States
(12) Patent Application Publication
(10) Pub. No.: US 2013/0052211 A1
(43) Pub. Date: Feb. 28, 2013

(54) VACCINES WITH ONCOFETAL ANTIGEN/ILRP-LOADED AUTOLOGOUS DENDRITIC CELLS AND USES THEREOF

(71) Applicants: Adel L. Barsoum, Mobile, AL (US); Suzanne Rohrer, Mobile, AL (US); Cindy C. Segers, Dothan, AL (US); Kurt M. Coggin, Mobile, AL (US)

(72) Inventors: Adel L. Barsoum, Mobile, AL (US); James W. Rohrer, Mobile, AL (US); Joseph H. Coggin, JR., Mobile, AL (US)

(73) Assignee: SOUTH ALABAMA MEDICAL SCIENCE, Mobile, AL (US)

(21) Appl. No.: 13/383,103

(22) PCT Filed: Jul. 9, 2010
(86) PCT No.: PCT/US10/41556

§ 371 Date: Aug. 15, 2012

Related U.S. Application Data
(60) Provisional application No. 61/270,570, filed on Jul. 9, 2009.

(51) Int. Cl. A61K 39/00; G01N 33/569; C12N 5/0784; A61P 37/04; A61P 35/00

(52) U.S. Cl. 424/184.1; 435/377; 435/374; 435/7.24

(57) ABSTRACT

Disclosed are compositions containing isolated monocyte-derived mature dendritic cells loaded with OFA/iLRP, or a fragment thereof that selectively stimulates T cytotoxic lymphocytes, and a carrier, vaccine compositions containing effective dosage amounts of the dendritic cells, methods of making the vaccines, and methods of cancer treatment or therapy that entail administration of the vaccines to cancer patients.

### CROSS-REFERENCE

The present application claims the benefit of the filing date of U.S. Provisional Patent Application No. 61/270,570, filed Jul. 9, 2009, the disclosure of which is incorporated herein by reference.

### BACKGROUND ART

The National Cancer Institute estimates 215,990 new cases of breast cancer diagnosis for the year 2004 in the United States with 40,110 disease related deaths (see the NCI website at www.nci.nih.gov/statistics). Although widespread screening to detect breast cancer at an early stage is employed today throughout America by using scheduled mammography, clinical breast examination, or both, it is still uncertain whether these measures indeed will decrease breast cancer mortality. The existence of such a benefit is uncertain mainly because of the inconsistency between studies. Therefore, it is expected that in the foreseeable future, advanced or metastatic breast cancer will remain a significant health problem in the US and worldwide.

In general, survival is dependent on the stage of the disease at the time of initial diagnosis but also on other tumor specific molecular characteristics. Although multimodality adjuvant and neo-adjuvant therapy has improved outcomes for earlier stage disease, unfortunately many breast cancers, especially locally advanced or stage III still will progress to metastatic or stage IV disease. The major improvement currently available to breast cancer patients today is the use of pre-surgery chemotherapy and radiation therapy. This stage of the disease is incurable with any kind of conventional treatment, as it only marginally impacts the overall survival. Median survival for de novo diagnosed stage IV breast cancer is between two and three years despite treatment, as various clinical trials have shown. Life expectancy for recurrent cancer presenting as stage IV following previous treatment given as adjuvant or neo-adjuvant therapy is even shorter, mainly because active therapies have already been used and may no longer be active upon their reintroduction. Although estrogen and/or progesterone receptor positive cancers often respond to repeated hormonal manipulations, the therapeutic benefit is often short-lived. The most activity chemotherapeutic compounds for breast cancer are anthracyclines and taxanes. Some tumors expressing Her2-neu may respond temporarily to a targeted monoclonal antibody, Herceptin. Once these compounds have been used and the disease has become refractory, prognosis is poor and considered to be less than one-year median survival.

Given the poor prognosis of advanced breast cancer, novel therapeutic approaches are needed to improve survival. Reliable, predictive laboratory assessments to monitor breast cancer progression or various specific conventional and experimental treatment regimens are not available. Given the fact that limits have been reached in optimizing schedules and dosing of currently available chemotherapeutic or hormonal agents (e.g., dose dense treatment), another avenue that has promise is immunotherapy. Although it is agreed that in principle, all cancers including breast cancers are amenable to immunotherapeutic approaches such as vaccination to stimulate autologous cell-mediated host immune responses, basic knowledge regarding strategies to achieve this goal is very limited. Dendritic cell (DC)-based breast cancer vaccines have received increasing attention and are currently being pursued by both the biotech/pharmaceutical industry as well as by academic researchers.^{1,2}

### SUMMARY OF THE INVENTION

The present invention relates to the use of oncofetal antigen (OFA)/immature laminin receptor protein (iLRP)-loaded autologous, mature, monocyte-derived DCs for treating cancer.

A first aspect of the present invention is directed to a method of producing an anti-cancer vaccine comprising autologous monocyte-derived oncofetal antigen (OFA)/immature laminin receptor protein (iLRP) loaded mature dendritic cells, comprising the steps of:

(a) collecting a sample of peripheral blood mononuclear cells (monocytes) from a cancer patient;

(b) extracting and purifying CD14+ monocytes from the collected sample;

(c) cultivating the CD14+ monocytes with GM-CSF in a medium containing GM-CSF and IL-4 under conditions effective to induce monocytes to dendritic cell differentiation;

(d) contacting the cultivated immature dentritic cells of (c), in the same, replenished or different medium with an amount of OFA/iLRP, or a fragment thereof that selectively stimulates T cytotoxic lymphocytes (and optionally a fragment that selectively stimulates T helper lymphocytes), under conditions and in an amount of OFA/iLRP effective to allow uptake and thus loading of the OFA/iLRP or fragment thereof by the dendritic cells;

(e) cultivating the OFA/iLRP-loaded dentritic cells of (d) with a dentritic cell maturation-inducing agent comprising a cocktail of cytokines under conditions and in amounts of the cytokines to allow the dendritic cells to mature; and

(f) harvesting the OFA/iLRP-loaded mature dendritic cells.

In some embodiments, the method entails the steps of (a) collecting peripheral blood mononuclear cells (monocytes) from a cancer patient (e.g., a breast cancer patient); (b) extracting or purifying CD14+ monocytes; (c) cultivating the CD14+ monocytes with GM-CSF (typically about 2000 to about 3000, e.g., about 2500, units per ml of medium) and IL-4 (typically in amounts of about 800 to about 1200, e.g., about 1000, units per ml of medium, to induce monocytes to dendritic cell differentiation (wherein cultivating is typically conducted for about 4 to about 6 days, e.g., about 5 days); (d) cultivating immature differentiated DCs with OFA/iLRP (or a fragment thereof, collectively “OFA/iLRP”), typically about 80 to about 120 nanograms per ml of medium, to allow uptake and thus loading of the OFA/iLRP by the dendritic cells; (e) cultivating the OFA/iLRP-loaded DCs with a DC maturation-inducing agent, typically a cocktail of cytokines (e.g., IL-1 (typically in an amount of about 8 to about 12, e.g., about 10 nanograms per ml of medium), IL-6 (typically in an amount of about 800 to about 1200, e.g., about 1000 units per ml of medium), INF-α (typically in an amount of about 8 to about 12, e.g., about 10 nanograms per ml of medium), and in preferred embodiments, the cocktail also containing PGE2 (typically in an amount of about 0.8 to about 1.2, e.g., about 1.0 micromolar) to allow the DCs to mature; (f) harvesting and then (g) cryopreserving the OFA/iLRP-loaded mature moDCs (which allows subsequent administrations of the vaccine without the need for additional harvestings of monocytes); (h) thawing and cultivating cryopreserved mature moDCs (typically about 2 days prior to administration, and in preferred embodiments, cultivated in the same type of medium); and (i) harvesting and resuspending cultivated moDCs in a suitable delivery vehicle or carrier, e.g., lactated Ringer's solution containing autologous plasma.

Another aspect of the present invention is directed to a composition, containing isolated monocyte-derived mature dendritic cells loaded with OFA/iLRP, or a fragment thereof that selectively stimulates T cytotoxic lymphocytes, and a carrier (e.g., a physiologically acceptable buffered medium).

Yet another aspect of the present invention is directed to a vaccine composition for use in cancer treatment or therapy, comprising an effective dosage amount of autologous, monocyte-derived mature dendritic cells loaded with OFA/iLRP or a fragment thereof that selectively stimulates T cytotoxic lymphocytes, and a pharmaceutically acceptable carrier. In some embodiments, the effective dosage amount is about 1×10⁷ viable dendritic cells. In some embodiments, the pharmaceutically acceptable carrier comprises lactated Ringer's solution, and optionally autologous plasma.

Yet a further aspect of the present invention is directed to a method of cancer therapy or treatment, comprising administering to a cancer patient a vaccine composition, comprising an effective dosage amount of autologous, monocyte-derived mature dendritic cells loaded with OFA/iLRP or a fragment thereof that selectively stimulates T cytotoxic lymphocytes, and a pharmaceutically acceptable carrier. In some embodiments, the patient has a solid tumor, such as breast cancer. Administration may be conducted daily, weekly, monthly or in some other embodiments once a month for three months. In some embodiments, the route of administration is intradermal. The method may also include various other steps that may be practiced before (e.g., premeditation) and/or after the vaccination.

An even further aspect of the present invention is directed to a method of monitoring the effect of cancer therapy, comprising:

(a) obtaining a blood sample from a cancer patient who has undergone treatment comprising administration of a vaccine composition comprising an effective dosage amount of autologous, monocyte-derived mature dendritic cells loaded with OFA/iLRP or a fragment thereof that selectively stimulates T cytotoxic lymphocytes, and a pharmaceutically acceptable carrier;

(b) cultivating CD4 and CD8 T-lymphocytes isolated from the blood sample in the presence of autologous, monocyte-derived mature dendritic cells loaded with OFA/iLRP isolated from the blood sample; and

(c) determining the frequency of gamma-interferon-secreting T lymphocytes and the frequency of Il-10-secreting T lymphocytes (and optionally the frequency of IL-4-secreting T lymphocytes), each or all relative to a control, wherein an increased frequency of the gamma-interferon-secreting T lymphocytes relative to a frequency prior to the treatment is indicative that the treatment is effective.

The above-mentioned and other features of this invention and the manner of obtaining and using them will become more apparent, and will be best understood, by reference to the following description.

### BRIEF DESCRIPTION OF THE DRAWINGS

FIG. 1 schematically shows a manufacturing scheme for OFA/iLRP-loaded, autologous, mature, moDCs.

FIGS. 2A and B are graphs showing immunization with intact OFA/iLRP-pulsed DCs, DCs pulsed with CTL-activating OFA/iLRP peptides, and DCs pulsed with OFA/iLRP peptides that activate IL-10-secreting, Ts cells reduced MCA1315 lung colony counts (A) and volume (B).

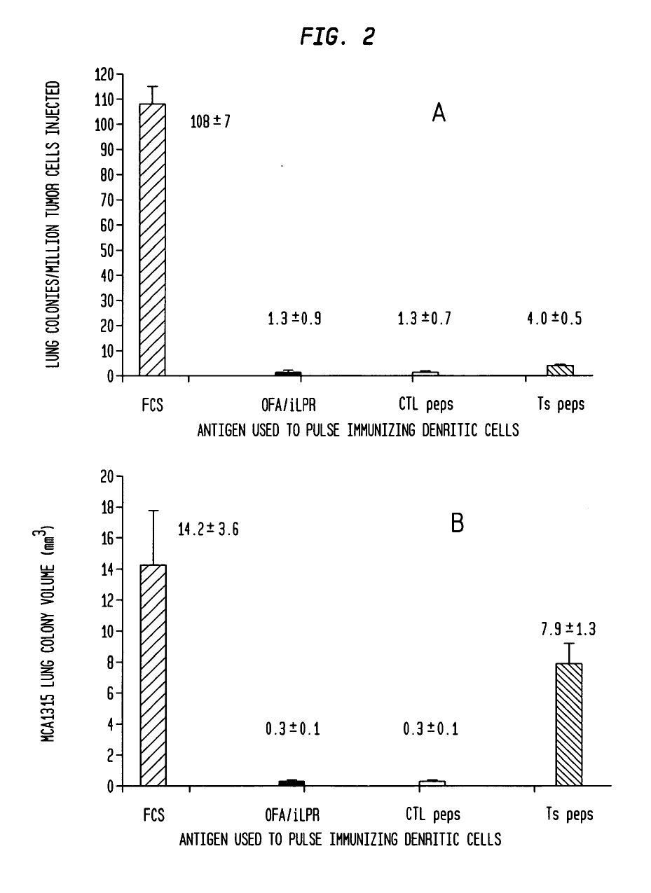

### BEST MODE FOR CARRYING OUT INVENTION

As used herein, “cancer” refers to a disease or disorder characterized by uncontrolled division of cells and the ability of these cells to spread, either by direct growth into adjacent tissue through invasion, or by implantation into distant sites by metastasis. Exemplary cancers include, but are not limited to, carcinoma, adenoma, lymphoma, leukemia, sarcoma, mesothelioma, glioma, germinoma, choriocarcinoma, prostate cancer, lung cancer (NSCLC and SCLC), breast cancer, colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer, esophageal cancer, gastric cancer, intestinal cancer, colon cancer, rectal cancer, myeloma, neuroblastoma, renal cancer (also known as renal cell carcinoma), and retinoblastoma. Thus, the invention embraces treatment of hematological cancers, soft tissue cancers and solid tumors, basically any cancer characterized by the synthesis of OFA by cancer cells. The cancer may be at any stage of its development, for example, the cancer may be a primary or metastatic cancer. The cancer patient may or may not have already undergone frontline therapy which was unsuccessful (e.g., it was not tolerated and was discontinued, or it was ineffective). Thus, the present invention may be useful as a frontline or second-line therapy such as in cases where the cancer is advanced (and wherein the patient received or did not receive frontline therapy, or wherein the cancer was refractory to frontline therapy.

As used herein, a “subject” or “patient” refers to an animal, including all mammals such as primates (particularly higher primates), sheep, dog, rodents (e.g., mouse or rat), guinea pig, goat, pig, cat, rabbit, cow, and human. In a preferred embodiment, the subject is a human. In another embodiment, the subject is an experimental animal or other animal suitable as a human disease model.

As used herein, “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

A subject to be treated may be identified in the judgment of the subject or a health care professional, and can be subjective (e.g., opinion) or objective (e.g., measurable by a test or diagnostic method).

An “effective amount” or “effective dosage amount” is an amount of a composition that is capable of producing a medically desirable result in a treated subject. The medically desirable result may be objective (i.e., measurable by some test or marker) or subjective (i.e., subject gives an indication of or feels an effect).

For example, a vaccine of the invention may be administered to a subject in need thereof via routes such as parenteral, e.g., intravenous, intradermal, subcutaneous, oral (e.g., inhalation), transdermal (topical), transmucosal, and rectal administration. Preferably, in the interest of breast cancer, the DCs will be injected intradermally with a single injection into the upper medial extremity close to the draining lymph nodes on the contralateral side of the original breast carcinoma lesion. The DCs may be admixed with a pharmaceutically acceptable carrier to form a pharmaceutical composition. “Pharmaceutically acceptable carriers” include solvents, dispersion media, coatings, antibacterial and antifungal agents, isotonic and absorption delaying agents, and the like, compatible with pharmaceutical administration.

Individual effective dosage amounts generally range from about 1×10⁵− to about 1×10⁹ (e.g., about 5×10⁵, about 1×10⁶, about 5×10⁶, about 1×10⁷, about 5×10⁷, about 1×10⁸, about 5×10⁸, about 1×10⁹₊), and more typically about (5×10⁶ to about 5×10⁷ cells, and even more typically about 1×10⁷ viable DCs) DCs may be used for each administration, which may be repeated daily, weekly, or monthly as needed (e.g., until remission). Preferably, treatment entails three monthly administrations. Preferably, about 1×10⁷ DCs are resuspended in 0.05 ml of lactated Ringer's solution containing about 0.4 to about 0.6% (v/v) autologous plasma.

In some embodiments, patients may be premeditated, e.g., with Benadryl® and Tylenol®, and observed in the oncology clinic for adverse side effects using monitoring of vital signs. Follow up may be performed one week and 4 weeks after each vaccination. Medical history as well as standard blood tests, coagulation tests, and urine analysis may be performed at each 4 week visit, additional testing as necessary. Standard DTH resting for recall antigens (tetanus toxoid, diphtheria toxoid, Streptococcus, tuberculin, Canclida albicans, Trichophyton-mentagrophytes, Proteus mirabilis; Multitest Immig-nost) may be performed before treatments start. The DTH response will be considered positive when at least one antigen induced induration and redness of greater than 2 mm in mean diameter by 48 hours. If they do not have at least one positive reaction, they will be considered to have a dysfunctional immune system and will be excluded from the study.

The patients may have CT scans before the first vaccination and every three months after the first vaccination to monitor the clinical status of the patient and her tumor. The patient may also have blood work done (typically monthly) using the acute panel, mineral panel, hepatic panel, and autoimmune profile to determine the patient's overall clinical status.

This invention exploits the universal tumor antigen oncofetal antigen/immature laminin receptor protein (OFA/iLRP) in a dendritic cell-based vaccine protocol. The protocol is designed to amplify and modify the inherent immune response in cancer patients (such as breast cancer patients) directed towards OFA/iLRP by actively vaccinating using autologous OFA/iLRP-pulsed dendritic cells reinjected into cancer patients.

OFA/iLRP has been found to be expressed in all human, as well as, murine cancers examined so far, which includes myeloid and lymphoid leukemias, lymphomas, renal cell carcinomas, prostate cancer, breast cancer, lung cancer, melanoma, squamous cell carcinoma, and ovarian cancer.^{12,18,22,26,27} It is not found on normal tissue after mid-gestation in fetal development.^{19,25} The 37 kDa OFA/iLRP is a highly conserved protein in humans, rodents, and other species.¹⁸⁻²² The 37 kDa OFA/iLRP upon binding laminin in the extracellular matrix induces secretion of metalloproteinases which digest the collagen in extracellular matrix. This process is important in tumor cell invasiveness, metastasis, and growth.²⁹ It has also been demonstrated that OFA/iLRP is essential to embryo cell invasiveness in normal embryo-fetal development and is matured after organogenesis into a non-immunogenic, dimeric 67 kDA mature laminin receptor protein.^{24,28} However, soon after transformation, cancer cells re-express the auto-immunogenic, 37 kDa OFA/iLRP form.^{22,30,31} That OFA/iLRP is not just a tumor marker, but is an immunogen that has been shown through experimental immunization of inbred mice with OFA/iLRP to result in dose-dependent induction of OFA/iLRP-specific cytotoxic T cells capable of killing syngeneic tumor cells.³² Similarly, immunization with syngeneic dendritic cells transfected with OFA/iLRP mRNA at weekly intervals induces significant antitumor immunity (67% of the mice are able to reject a lethal dose of A20 lymphoma cells).²⁶ Also, OFA/iLRP-specific memory Th1 and cytotoxic T cells are clonable from the spleens of long-term survivors of x-irradiation induced lymphomagenesis in RFM strain mice without any experimental manipulation whereas, age-matched non-irradiated controls have no memory T cells specific for OFA/iLRP.

OFA/iLRP is immunogenic in humans also. In vitro stimulation of PBMC from breast carcinoma patients with autologous x-irradiated breast carcinoma cells generates CD4 and CD8 OFA/iLRP-specific T cell clones which are capable of killing the autologous tumor cells.¹⁷ Approximately 32% of the autologous tumor-reactive T cell clones were specific for OFA/iLRP.¹⁷ In a recent study, OFA/iLRP was shown to be expressed in a number of hematologic cancer lines, but not in normal human monocytes, DCs, and T cells from healthy individuals. In vitro stimulation of naive T cells from healthy volunteers with autologous DCs which have been transfected with tumor RNA induced OFAALRP-specific cytotoxic T lymphocytes capable of specific killing of AML and CLL blasts, but showed no killing of OFA/iLRP-negative, diploid CD34 progenitor cells, bone marrow cells, normal B lymphoblasts, and dendritic cells.²⁶

By producing dendritic cells from a cancer patient's blood monocytes, loading them with OFA/iLRP, and inducing their maturation with a cocktail of cytokines, an OFA/iLRP-specific autologous cellular therapy for the treatment of carcinoma (e.g., breast cancer or metastatic breast cancer) is produced. Without intending to be bound by theory, the mechanism of action of the OFA/iLRP-loaded, autologous, mature, monocyte-derived dendritic cells (moDCs) is that of an active immunotherapy to generate a cancer-specific immune T cell response that will fight the patient's cancer. The OFA/iLRP-loaded mature, moDCs do not have a direct cytotoxic effect. Instead, the anti-tumor effect is generated by the presentation of OFA/iLRP and activation of effector T lymphocytes specific for OFA/iLRP, which are present in the cancer patient's blood and lymph.¹⁷ The activated OFA/iLRP-specific T cells then mount an attack against the carcinoma cells (which are OFA/iLRP-expressing). This mechanism of action is believed to be unlike chemotherapeutic drugs that directly kill the tumor cells. It is also believed to be different from immunotherapies that generically stimulate the immune response, such as IL-2, or specifically target the tumor via an anti-tumor antibody (herceptin). Because the product requires the development of an immune response after administration, there is some delay in the potential effect of the product; the generation of the immune response and a clinical effect of that immune response may take several weeks.

Examples of OFA/iLRP fragments that specifically stimulate Tc lymphocytes and fragments that specifically stimulate Th lymphocytes are disclosed in U.S. Patent Application Publication 200610165709, the relevant portions of which are herein incorporated by reference.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### General Investigational Plan

Primary Objectives
Determine safety and toxicity of 3 monthly-repeated vaccinations with OFA/iLRP-loaded autologous dendritic cells in patients with metastatic breast cancer
Determine immunological responses to the vaccine by measuring DTH response and induction of OFA/iLRP-specific T-cells

Secondary Objectives
Evaluate objective clinical responses
Evaluate time to disease progression
Evaluate survival

Study Overview

The study is an open-label study to assess safety and immune responses to the universal tumor antigen OFA/iLRP. All patients will be immunized with 1×10⁷ viable OFA/iLRP-loaded mature, autologous monocyte-derived DCs. The DC vaccine will be administered intradermally into the proximal medial upper extremity, contralateral to the original site of breast cancer once every month for 3 months. Patients who initially show clinical amelioration of their cancer, but subsequently begin to show progressive disease may, at the discretion of the oncologist, be re-immunized with 3 more monthly intradermal injections of the OFA/iLRP-loaded, mature autologous monocyte-derived dendritic cell vaccine. Changes in the tumor will be documented. The patient will remain in the study unless toxicity or adverse side effects require discontinuation following RECIST and CTC guidelines, or if the patient withdraws for any other reason.

We have decided to inject 10⁷ viable OFA/iLRP-loaded autologous, mature, moDCs intradermally in the medial upper extremity contra-lateral to the original breast carcinoma lesion 3 times at monthly intervals.

Protocol

Protocol Synopsis

Study Title: A Phase I/TI Vaccine Study with autologous Dendritic cells loaded with Oncofetal Antigen/iLRP, in Patients with Metastatic Breast Cancer Vaccine: Autologous dendritic cells expanded ex vivo and loaded with Oncofetal Antigen/iLRP. The following study and all preparatory and tangential aspects thereof, described below using the present or future tenses, are now underway.

Objectives:

Primary: Determination of safety and tolerability and immunological responses of a vaccine comprised of ex vivo cultured OFA/iLRP-loaded autologous dendritic cells in advanced, metastatic breast cancer patients.

Secondary: Evaluation of clinical responses as measured by physical examination and imaging studies (CT/MRI) and correlation to immunologic responses by the breast cancer patients. Subject survival also will be measured as a secondary endpoint.

Study Design: Prospective open study

Patients: Metastatic breast cancer patients after at least one failed prior cytotoxic therapy. Age: >18 years. Performance status: ECOG 0 and 1. Adequate bone marrow, renal, liver and heart function. Signed written-informed consent.

Sample Size: 27 patients total.

Safety Criteria: Common Terminology Criteria for Adverse Events version 3.0 (CTCAE) of the National Cancer Institute

Efficacy Criteria: For clinical response assessment the “Response Evaluation Criteria in Solid Tumors (RECIST)” will be used.

Immunological evaluation to include assessment of immunological response by DTH skin test reaction and specific T-cell responses to OFA/iLRP in vitro will be used.

Duration: All patients are to receive three intradermal injections of the vaccine at monthly intervals. Patients who after their initial 3 vaccinations show clinical amelioration of their cancer, but subsequently begin to show progressive disease may, at the discretion of the oncologist, be re-vaccinated with 3 more monthly intradermal injections of the OFA/iLRP-loaded, mature, autologous, monocyte-derived DC vaccine. Disease progression will be documented and the patient's immune status monitored for 2 years following the initial vaccine administration, until the patient requests discontinuation, or until observed toxicities require termination of the protocol.

Statistics: This is an open study with no placebo control. Statistical analysis will be performed for immunological parameters over time compared to baseline for the entire population as well as the analysis between the OFA/iLRP-loaded, autologous, mature DC vaccine-injected patients and historical controls.

Study Schedule

TABLE I
(Study Flow Chart)

| | Pm | On study (days) | | | | | Every 3 | |
|---|---|---|---|---|---|---|---|---|
| | Vaccination | DO | D7 | D14 | D21 | D28* | months | Off study |
| Informed consent | x | | | | | | | |
| Inclusion/exclusion | X | | | | | | | |
| evaluation | | | | | | | | |
| Blood samples′ | X | x | | | | x | X | x |
| Physical exam² | x | x | X | | | x | x | x |
| CT of brain² | x | | | | | | | |
| Radiographic Tumor | x | | | | | | x | X |
| evaluation′ | | | | | | | | |
| (CT/111:RD | | | | | | | | |
| Blood samples for | X | | | | | X | X | |
| T-Cell response | | | | | | | | |
| Isolation of | X | | | | | | | |
| dendritic cells | | | | | | | | |
| Vaccinations | | X | | | | X** | | |
| Adverse Event | | X | X | | | X | X | X |
| Cell harvest for | X | | | | | | | |
| next vaccination | | | | | | | | |
| Tumor sample | X | | | | | X | | |
| if | | | | | | | | |
| accessible | | | | | | | | |
| DTI-P | X | | | | | X | | |

*D28 = DO (1 cycle = 28 days)
**Vaccination will be administered once a month for 3 months.

1) The blood test results will not be older than 2 weeks: Acute panel, mineral panel and hepatic panel and autoimmune profile (including rheumatoid factor and ANA profile) (hepatitis B and C, HIV and 1NR at baseline only; pregnancy test within the last 14 days)
2) Vital signs, and ECOG performance status
3) CT of the brain not older than 4 weeks
4) CT of chest, abdomen, and pelvis will not older than 4 weeks. Patients will be radiographically restaged in 2 month 1.0 intervals, upon termination of the protocol, or if clinically indicated otherwise.
5) 50 ml ACD-blood
6) Vaccination to start within 3 weeks after study inclusion
7) DTH skin reaction to common recall antigens (see below) before inclusion in study, and DTH reaction from the vaccine to be registered and recorded by the patient 48 hours after each vaccination.
8) Tumor biopsy sample from easily accessible tumor tissues such as cutaneous/subcutaneous or single lymph node metastasis, to assess OFA/iLRP expression status and infiltrating T cell populations, if possible repeated after 3 months.

Patient Selection

All patients will be treated in an oncology clinic.

Patient cells will be cultured and treated ex vivo at Laboratories.

Eligibility Criteria

ALL of the following conditions must apply to the prospective patient at screening prior to receiving study agent:
1. Stage 1V histologically proven breast cancer as defined by the AJCC Cancer Staging Manual (6th. Edition 2003).
2. Patients may be hormone receptor (ER/PR)-positive or negative. However, hormone receptor-positive patients should have failed at least one hormonal therapy prior to enrollment.
3. Patients may be HER-2-negative or HER-2-positive (defined as HER-2 3+ positive by Hercept test, otherwise positivity has to be proven by FISH).
4. Patients must have completed at least one prior form of chemo- and/or radiation therapy for their disease and have failed to achieve remission or have progressed following this treatment. The chemotherapy must have been either anthracycline or taxane-based or a combination thereof. HER-2-positive patients may have also been treated with HER-2-targeted therapy either alone or in combination with chemotherapy. A limit of up to three prior chemotherapy (with or without biologics)-based regimens for metastatic disease will be permitted.
5. Patients with excessive tumor burden will not be eligible. Estimated tumor burden has to be less than 125 cm³.
6. There must be no clinical or radiographic signs of active brain metastases (CT of brain), or disease to the brain that is not considered controlled.
7. At least 4 weeks must have elapsed since chemotherapy or biological therapy and 2 weeks must have elapsed since radiotherapy.
8. Female patients must be at least 18 years of age, must be ambulatory with a ECOG performance status of <2, must have common recall antigen DTH skin reaction >2 mm, must have lab values as following: ANC >1.5×10⁹/L, platelets>100×10⁹/L, Jib>9 g/dL, creatinine<1.8 mg/dL− or a creatinine clearance>35 mL/min, total bilirubin<2 the upper limit of normal, AST and ALT<2.5 the upper limit of normal, albumin>2.5 g/L.
9. If of child bearing potential, patients must practice a reliable method of contraception at screening and must agree to continue this status until 6 months after receiving the last study vaccine injection. An HCG (pregnancy) test will be done monthly until the 3 vaccinations are complete.
10. Signed informed consent is to be obtained according to ICH-GCP guidelines before the patient is subjected to any extra diagnostic procedures performed for evaluation of eligibility for the trial.

Exclusion Criteria
1. History of other prior malignancy, with the exception of curatively treated basal cell or squamous cell carcinoma of the skin or cervical cancer stage
2. Active infection requiring continuous use of antibiotic therapy
3. Significant cardiac or other medical illness that would limit activity or survival, such as severe congestive heart failure, unstable angina, or serious cardiac arrhythmia
4. Autoimmune disease currently treated with steroids
5. Adverse reactions to vaccines such as anaphylaxis or other serious reactions, e.g., life-threatening reactions to medicine
6. History of immunodeficiency or autoimmune disease such as rheumatoid arthritis, systemic lupus erythematosus, scleroderma, polymyositis-dermatomyositis, juvenile onset, insulin-dependent diabetes, or a vasculitic syndrome
7. Pregnancy or lactation
8. Any reason why, in the opinion of the investigator, the patient should not participate
9. Patients who have received cytoxic anti-tumor therapy within 4 weeks prior to vaccination.
10. Patients with active hepatitis (B, C) or HIV-positive individuals

Withdrawals

Patients are free to withdraw from the study at any time without the need to give reasons, and without prejudice to further treatment. Patients may be withdrawn from the study at any time at the discretion of the investigator.

If possible, a final clinical assessment will be made of any withdrawing or withdrawn patients. The reason for discontinuation will be recorded. The investigator is obliged to follow any significant adverse events until the outcome is assessable.

Concomitant Medication

No other concurrent anti-breast cancer therapy will be permitted with the exception of biphosphonates for continuous treatment of bone metastasis, if present. Hormone receptor-positive patients must have failed at least one line of hormonal therapy and discontinued it for the duration of the study participation.

All concomitant medication, or medication administered during the study period must be reported in the case record form (CRF) in the space reserved for this purpose, noting the type of medication, the dose, duration and indication. Any changes in documented, permitted concomitant medication already being taken at the beginning of the study must also be recorded.

Particular attention should be paid to existing drugs or treatments that could influence the intended effects or mask side effects of the experimental treatment.

Continuation of patients on the protocol who for any reasons unrelated to the study need treatment with systemic corticosteroids will be decided on a case to case basis as to whether continuation on protocol is of benefit for the patient or whether the patient be replaced by new patients.

Treatment

Treatment Cohorts

A total of 27 patients will be accrued. The protocol is designed as a seamless Phase I/II study. The first 3 patients will comprise the phase I study and if the vaccine is shown to be safe using NCI-defined CTC criteria (see the website at ctep.cancer.gov/forms/CTCAEv3.pdf), they and the remaining 24 patients will comprise the phase II study. The statistical reasons for using 27 patients in this study is outlined in the Statistical Analysis section. Expected side-effects include only mild flu-like symptoms, fever, and erythema and induration at the site of vaccination as a side effect. Given its universal presence on all tumors examined to date,^{22,30,31} immunity to it induced during normal pregnancy, and the fact that breast carcinoma patients have OFA/iLRP-specific memory effector and regulatory T cells in their blood without any experimental manipulation,¹⁷ we do not expect any adverse effects from the vaccination with autologous mature moCDs loaded with OFA/iLRP. We propose to use a moderate dose of DC using the intradermal route (1×10⁷).¹² Toxicity will be monitored using the NCI CTC criteria (see the website at ctep cancer. gov/forms/CTCAE 3.p df).

Dendritic Cell Vaccination

Vaccination will start in patients within 3 weeks after inclusion in the trial. Patients are to be treated as outpatients. After each vaccination the patients will remain at the Clinic for 1 hour, a monitoring period for safety reasons. As for all vaccinations, anaphylactic shock is a possible risk, however so far no serious toxic reactions have been reported in similar studies. To minimize this risk, the monocyte-to-dendritic cell differentiation will be performed in serum-free medium (CellGro-DC medium) optimized for this differentiation. This will mean that NO IgE will be present during this preparation used for immunotherapy. Also, cryopreservation of the antigen-loaded, mature DCs will be done in autologous serum and the DCs will be administered in lactated Ringer's solution with 1% autologous serum.

Two days before vaccination, the OFA/iLRP-loaded, autologous, mature monocyte-derived DCs which had been cryopreserved in liquid nitrogen, will be rapidly thawed and washed in CellGro-DC serum-free medium and cultured 48 hours at 37° C. in a humidified 95% air/5% CO₂ atmosphere. A sample of the DCs will then be counted for viability using the Trypan blue dye exclusion method and, if >70% viable, will be resuspended in 500 g/l of lactated Ringer's solution with 1% autologous serum. Cells will be injected into the upper extremity contra-lateral to the original site of the breast cancer into an intradermal area close to the draining axillary lymph nodes.

Vaccination Schedule

Patients will be vaccinated with autologous mature, OFA/iLRP-loaded moDCs once every month for 3 months.

Dose Finding:

In principal tumor lysate based DC clinical vaccinations appear to have little toxicity at various DC doses that were tested.¹² Likewise, no significant toxicity was detected over 40+ months post immunotherapy with purified OFA/iLRP-loaded autologous, mature moDCs. It is hypothesized that adverse events with DC based vaccinations is not dose dependent. Although the investigators support this hypothesis, it may not be applicable to all different proteins employed for vaccinations. Since we propose testing of a unique and highly conserved protein, which appears to be an immunogenic universal tumor protein,²² we propose toxicity evaluation as part of this protocol.

Anticipated Risks of Particular Severity

More than 60 different DC vaccination trials with almost 1000 patients have been published in recent years.¹⁶ Uniformly, the reported toxicity from these studies has been modest,^{33,40} mostly transient fever, local reactions and the induction of autoimmune vitiligo. Serious systemic toxicity has not been reported.

Peripheral blood draws pose minimal risks. In some patients, with poor venous access, it may be necessary to insert central access in form of a port or PICC line, with slightly increased risk, although still considered minimal as it will be performed by professionals competent in these procedures. Potential complication associated with these more invasive procedure include bleeding, infections and pneumothorax. The risks with the collection procedure are similar to those involved in blood donation and may in rare cases include nausea, vomiting, fainting or dizziness, hematoma, seizures, blood loss, infection and in some cases nerve damage. This will be minimized by administration of intravenous fluids (normal saline) prior to the collection procedure.

It is theoretically possible that any form of vaccination protocol can lead to clinically significant autoimmune diseases. Patients will be monitored for such occurrence using laboratory testing.

It is also theoretically possible that tumor vaccination could lead to the opposite of the desired therapeutic effect that is creating tumor tolerance. This could happen by inducing expansion of T-suppressor cell clones. It is possible, that this could accelerate disease progression. There are no data in the literature that have previously described such a phenomenon.

DTH Test:

The patient or a responsible caregiver will receive instruction for measuring the local response at the injection site to cover the interval between office visits. After each intradermal DC vaccine injection, the DTH reaction at the injection site will be recorded daily by the patient or a caregiver after 48 hrs. The largest diameters of the erythema will be recorded in a provided diary card to be mailed back to the investigator or the clinical nurse. The DTH test will be considered positive if the largest diameter of the skin reaction has a diameter>5 ram.

Stopping Rules

Review of safety events will be performed by the Drug Safety Monitoring Board (DSMB), appointed by the Providence Hospital IRB which will oversee the progress of the study on an ongoing basis. The first safety analysis will be performed after 3 patients have been enrolled and treated at least once. Subsequent analysis will be performed every 3 months.

Subjects will be withdrawn although continued to be followed by the investigators to determine long term toxicity and assess disease status from the study if any of the following occurs:
1. Excessive toxicity at least “probably related” to the study drug or procedure. This includes:
   Any allergic/hypersensitive toxicity Grade 2 or higher (per NCI-CTC version 3.0) including asymptomatic bronchospasm
   Any toxicity of Grade 3 or higher (except alopecia) not related to progressive disease
   Other study vaccine related toxicity that results in premature termination of therapy
2. Administration of other therapeutics for breast cancer including systemic steroid or other immunosuppressive therapy.

If the incidence of the above drug-related events designated as excessive toxicity is greater than 33% within one cohort, dosing will cease. This applies to any procedure-related toxicity observed greater than Grade 3 or Grade 2 for allergic reactions. Decision for continuation or modifications to treatment may occur after discussion with the Drug Safety Monitoring Board. Re-evaluation will occur after each PROBABLY RELATED grade 3 or allergic grade 2 toxicity.

Vaccination will be stopped if disease progression occurs, using RECIST criteria, significant toxicity occurs as determined by monitoring following CTCAE v3.0 of the National Cancer Institute (see the website at ctep.caneer.goc/forms/CTCAEv3.pdf), or the patient withdraws his consent to continue with the treatment.

Tumor Sampling

Easily accessible tumor tissues will be sampled before and after each treatment to measure the expression of OFA/iLRP by immuno-histochemistry staining and flow cytometry as well as to characterize infiltrating T cell populations, and if possible to establish tumor cell lines for further studies. Any additional tissue will be frozen and stored in the Laboratory of Molecular Biology Biobank of the USA Main Campus.

Pre-Study

All eligibility criteria should be assessed together with relevant baseline parameters prior to study inclusion (inclusion/exclusion criteria).

Clinical Status:

Case history, physical examination, ECOG performance status and MRI or CT

Laboratory Analysis:

Complete blood count, acute care, hepatic- and mineral panels before vaccination and HIV, Hep B and C, INR, pregnancy test within the last 14 days. Autoimmune profile: Anti-DNA (double-stranded) antibodies; antinuclear antibodies; Rheumatoid factor; complement C3 will be done before vaccination to obtain baseline levels and also at 1 month after vaccination and then every 3 months thereafter to monitor changes which might result from the vaccinations.

Immunological Tests:

Blood samples (50 ml) will be taken to establish baseline values for OFA/iLRP specific T-cells and antibody. Details of the methodology of these tests are described below.

On-Study:

Weeks 1 and 4, and then monthly until end of study

Vaccination and DTH test:

The patients will be injected intradermally with 1×10⁷ autologous, mature monocyte-derived dendritic cells suspended in 50 μl of lactated Ringer's solution supplemented with 0.5% autologous plasma over 5 minutes in the upper extremity on day one.

A DTH response (induration and redness) at the injection site will be read 48 hours later by the patient or a designated caregiver. This DTH response will be a measure of the patient's specific cell-mediated immune response to OFA/iLRP. This will be done for each immunization injection.

Clinical Status:

During treatment patients will have a complete blood count, acute care-, hepatic- and mineral panels as well as an auto immune profile before each vaccination. In addition, a short history and physical examination, including vital signs, ECOG performance status.

Adverse Event:

Any adverse event reported by the patient and/or by the physician will be recorded.

Follow-up of the patient in the hospital 1 hour after vaccination Radiographic Evaluation:

Tumor specific radiographs will be obtained every 3 months (CT scan chest and abdomen) or sooner if clinically indicated.

End-of-Study:

Clinical Status:

Complete history and physical examination. Complete evaluation of evaluable lesions with physical examination and appropriate X-rays and/or scans will be done two-three weeks after the last immunization. Arrange for new diagnostic examinations, CT and X-ray, every 3rd month. Complete blood count, acute care, hepatic and mineral panels and autoimmune profile will be obtained.

Adverse Event:

Any adverse event reported by the patient and/or by the physician will be recorded.

T-Cell Response:

To obtain lymphocytes for the T-cell assays, peripheral blood (50 ml) will be drawn.

Off-Study

Unacceptable toxicity as defined in Stopping Rules section or as determined by the investigator, will exclude the patient from the study. An off-study evaluation of any parameters will be conducted within seven days, if possible, after withdrawal from the study. Disease progression or ECOG status deterioration will also terminate the study, unless continuation of the program would be found otherwise to be of clinical benefit to the patient.

Follow-Up

Patients completing the protocol will be followed-up every 3 months with new CT examinations and blood samples. Long-term follow-up, for the first five years, will be done according to standard practice for this patient group.

Adverse Events

Common Terminology for Adverse Events v3.0 (CTCAE) is provided on the Internet at ctep.cancer.gov/forms/CTCAEv3.pdf). In the recording of adverse events, the investigator must observe the following definitions.

An Adverse Event is:

At baseline: Any present existing undesirable experience occurring to a patient whether or not considered related to a pharmaceutical product

During the study: Any undesirable experience occurring to a patient during this clinical study, whether or not considered related to the investigational product

Adverse events will be spontaneously reported by the patient, observed by the investigator, or elicited by the investigator by asking the patient specific questions according to a defined scheme.

If the patient has experienced adverse event(s), the investigator will record the following information in the CRF:
The nature of the event(s) will be described by the investigator in precise standard medical terminology (i.e., not necessarily the exact words used by the patient).
The duration of the event will be described in terms of the time from onset and the number of hours/days affected.

The intensity of the adverse event will be described according to CTC adverse event grading, or as mild (grade 1), moderate (grade 2) or severe (grade 3):
Mild: The adverse event causes minimal discomfort and does not interfere in a significant manner with the patient's normal activities.
Moderate: The adverse event is sufficiently uncomfortable to produce some impairment of the patient's normal activities.
Severe: The adverse event is incapacitating, preventing the patient from participating in his/her normal activities.

The causal relationship of the event to the study medication will be assessed as one of the following:

Unlikely: The adverse event:
does not follow a reasonable temporal sequence following administration of the drug (treatment)
could readily have been produced by the patient's clinical state, environmental or toxic factors or other medication administered to the patient
does not follow a normal response pattern to the suspected drug (treatment)

Possible: The adverse event:
follows a reasonable temporal sequence following administration of the drug (treatment)
follows a known response pattern to the suspected drug (treatment)

Probable: The adverse event:
follows temporal sequence following administration of the drug (treatment)
could not be reasonably explained by the patient's clinical state, environmental or toxic factors or other medication administered to the patient
follows a known pattern or response to the suspected drug (treatment)

The outcome of the adverse event is whether the event is resolved or still ongoing. Significant adverse events must be followed up by the investigator to a satisfactory conclusion whenever possible. Details of the follow-up should be documented (i.e., if treatment is required, if hospitalization is required, etc.).

Serious or Unexpected Adverse Events

A serious adverse event means an adverse experience that is fatal, life-threatening, disabling or which results in in-patient hospitalization or prolongation of hospitalization. In addition, congenital anomaly and occurrence of malignancy are always considered serious adverse events.

If the adverse event is defined as serious, the event must be reported to the regulatory agency in accordance with national regulations. A special form (Report of Adverse Events) has to be used for this purpose.

Adverse Event Reporting Requirements

Adverse events are undesirable signs/symptoms or events that occur during study participation whether or not causally related to study drug or treatment. All adverse events will be recorded in the patient's medical records and on the data collection documents (CRF).

The investigator has an obligation to report any adverse events to the IRB, DSMB and the FDA and if applicable, according to the following deadlines:
a) Fatal or life-threatening adverse drug reactions that are unexpected must be reported immediately, (e.g., by telephone, facsimile, or in writing) no later than 7 calendar days after the event in question followed by as complete a report as possible within 8 additional calendar days. The report should include an assessment of the importance and implication of the findings, including relevant previous experience with the same or similar medicinal products.
b) Serious and unexpected adverse drug reactions must be reported within 15 days.
c) All serious adverse events must be reported collectively in an annual report.
d) All adverse events must be reported collectively in a final report.

The reports on adverse drug reactions under a) and b) above must be accompanied by an account of any interruption in the treatment or any unblinding of the treatment code if applicable, the investigator's assessment of the causal relationship, and the consequences for the trial.

Non-Serious Adverse Events

Only minor adverse events as heating, red skin and stinging have been reported in other studies with similar vaccines. The patient will be instructed to record all adverse events in his/her study diary. The physician will further document events within a special adverse event form.

All adverse events will be recorded in the patient's CRF.

Assessment of Responses

Assessment of Immunological Responses

The DTH-test will be considered positive if the area of the skin reaction has a diameter>5 mm.

The following parameters of immune response will be recorded:
1. In vivo presence or absence of a DTH response, the point of time of appearance of a positive DTH response, and the size of the DTH response;
2. In vitro immune responses as recorded in the ELISPOT assay to determine vaccination-induced changes in sub-typing of OFA/iLRP-specific T-cell subclasses detected (i.e., HLA-bound OFMIRP peptide-induced differential cytokine responses, of CD4 and CD8 T cells); and
3. In vitro anti-OFA/iLRP IgG and IgM titers determined by OFA/iLRP ELISA.

The methodology of the assays mentioned in parameters 2 and 3 are discussed below.

Assessment of Antitumor Responses

Although response is not the primary endpoint of this trial, patients with measurable disease will be assessed by standard criteria for response. Baseline scans, including CT of chest, abdomen and pelvis may not be older than 4 weeks prior to beginning immunotherapy. Patients will have examinations repeated every third month; if response is documented, verification CT scan not less than 6 weeks later will be performed.

### DEFINITIONS

Response and progression will be evaluated in this study using the new international criteria proposed by the Response Evaluation Criteria in Solid Tumours (RECIST) CIST) Committee (see the web site at ctep.cancer.gov/guidelines/recist.html).⁶⁰ Changes in only the largest diameter (one-dimensional measurement) of the tumor lesions are used in the RECIST

criteria. Note: Lesions are either measurable or non-measurable using the criteria provided below. The term “evaluable” in reference to measurability will not be used because it does not provide additional meaning or accuracy.

Measurable Disease

Measurable lesions are defined as those that can be accurately measured in at least one dimension (longest diameter to be recorded) as >20 mm with conventional techniques (CT, MRI) or as >10 mm with spiral CT scan. All tumor measurements must be recorded in millimeters (or decimal fractions of centimeters).

Non-measurable Disease

All other lesions (or sites of disease), including small lesions (longest diameter<20 mm with conventional techniques or <10 mm using spiral CT scan), are considered non-measurable disease. Bone lesions, leptomeningeal disease, ascites, pleurallpericardial effusions, lymphangitis cutis/pulmonis, inflammatory breast disease, abdominal masses (not followed by CT or MRI), and cystic lesions are all non-measurable.

Target Lesions

All measurable lesions up to a maximum of five lesions per organ and 10 lesions in total representative of all involved organs will be identified as target lesions and recorded and measured at baseline. Target lesions will be selected on the basis of their size (lesions with the longest diameter) and their suitability for accurate repeated measurements (either by imaging techniques or clinically). A sum of the longest diameter (LD) for all target lesions will be calculated and reported as the baseline sum LD. The baseline sum LD will be used as reference by which to characterize the objective tumor response.

Non-Target Lesions

All other lesions (or sites of disease) will be identified as non-target lesions and also be recorded at baseline. Non-target lesions include measurable lesions that exceed the maximum numbers per organ or total of all involved organs as well as non-measurable lesions. Measurements of these lesions are not required but the presence or absence of each will be noted throughout follow-up.

Guidelines for Evaluation of Measurable Disease

All measurements will be taken and recorded in metric notation using a ruler or calipers. All baseline evaluations will be performed as closely as possible to the beginning of treatment and never more than 4 weeks before the beginning of the treatment.

The same method of assessment and the same technique will be used to characterize each identified and reported lesion at baseline and during follow-up. Imaging-based evaluation will be used in preference to evaluation by clinical examination when both methods may be used to assess the antitumor effect of a treatment.

Clinical Lesions

Metastatic breast cancer lesions will only be considered measurable when they are superficial (e.g., skin nodules and palpable lymph nodes). In the case of skin or subcutaneous lesions, documentation will include a ruler to estimate the three-dimensional size of the lesion.

Conventional CT and MRI

These techniques will be performed with cuts of 10 mm or less in slice thickness contiguously. Spiral CT will be performed using a 5 mm contiguous reconstruction algorithm. This applies to tumor within of the chest, abdomen, and pelvis. Tumor within head and neck and extremities typically will require more specific protocols.

Cytology, Histology

These techniques may be used to help differentiate between partial responses (PR) and complete responses (CR) in certain cases.

Anti-Tumor Response Criteria Evaluation of Target Lesions:

Complete Response (CR): Disappearance of all target lesions.

Partial Response (PR): At least a 30% decrease in the sum of the longest diameter (LD) of target lesions, taking as reference the baseline sum LD.

Progressive Disease (PD): At Least a 20% increase in the sum of the LD of target lesions, taking as reference the smallest sum LD recorded since the treatment started or the appearance of one or more new lesions.

Stable disease (SD): Neither sufficient shrinkage to qualify for PR nor sufficient increase to qualify for PD, taking as reference the smallest sum LD Since the treatment started.

Evaluation of Non-Target Lesions

Complete response (CR): Disappearance of all non-target lesions and normalization of tumor marker level.

Incomplete Response:
Stable Disease (SD): Persistence of one or more non-target lesion(s) and/or maintenance of tumor marker level above the normal limits.
Progressive disease (PD): Appearance of one or more new lesion(s) and/or unequivocal progression of existing non-target lesions.

Although a clear progression of “non-target” lesions only is exceptional, in such circumstances, the opinion of the treating physician should prevail, and the progression status should be confirmed at a later time by the principal investigator.

Evaluation of Best Overall Anti-Tumor Response

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

Note: Patients with a global deterioration of health status requiring discontinuation of treatment without objective evidence of disease progression at that time will be reported as “symptomatic deterioration”. Every effort will be made to document the objective progression even after discontinuation of treatment. In some circumstances, it may be difficult to distinguish residual disease from normal tissue. When the evaluation of complete response depends upon this determination, the residual lesion preferably will be investigated (fine needle aspirate/biopsy) before confirming the complete response status.

Confirmatory Measurement/Duration of Anti-tumor Response Confirmation

To be as Signed a status of PR or CR, changes in tumor measurements will be confirmed by repeat assessments that should be performed at a minimum 4 weeks-after the criteria for response are first met. In the case of SD, follow-up measurements must have met the SD criteria at least once after study entry at a minimum interval of 4 weeks.

Duration of Overall Response

The duration of overall response will be measured from the time measurement criteria are met for CR or PR (whichever is first recorded) until the first date that recurrent or progressive disease is objectively documented (taking as reference for progressive disease the smallest measurements recorded since the treatment started). The duration of overall CR will be measured from the time measurement criteria are first met for CR until the first date that recurrent disease is objectively documented.

Duration of Stable Disease

Stable disease will be measured from the start of the treatment until the criteria for progression are met, taking as reference the smallest measurements recorded since the treatment started.

Duration of Response

The duration of partial response or stable disease will date from the commencement of treatment until the documentation of progression; the duration of complete remission will date from the moment the complete response has been documented.

Duration of Survival

Overall survival in the study will be dated from the start of treatment until death or last follow-up. Progression-free survival will be calculated from the start of treatment to the date of documented disease progression.

Assessment of Clinical State

Assessment of clinical state includes a general physical examination and a carefully planned patient interview with evaluation of the ECOG performance status. Deviation from expected laboratory tests, subjective complaints or abnormal physical findings will be recorded and will lead to appropriate investigations according to general hospital routines for this patient category. Assessment of clinical state will be performed by one of the participant clinical doctors. In case of acute medical problems outside normal working hours, the patient will be instructed to contact the hospital.

Statistical Analyses

The study is a phase trial with a non-randomized phase II portion.

Statistical analysis will be performed on laboratory parameters. Our research indicates that it is very unlikely that the number of women in the historical control⁶⁹⁻⁷¹ that had time to progression greater than 5 months would have been in excess of 15%. We hypothesize that at least 35% of the women will have time to progression greater than 5 months after our experimental vaccine therapy described herein. Our required sample size is 27 women; eight or more women must have time to progression greater than 5 months for us to accept our hypothesis. Given that the true time to progression rate in this is indeed 35%, we will have at least an 80% power to correctly accept the hypothesis and a maximum 5% chance of erroneously concluding that the new therapy time to progression is no different than the historical control.

### LIST OF ABBREVIATIONS

AE=adverse event
CRF=case record form
DTH=delayed type hypersensitivity
HLA=human leukocyte antigen
INR=international normalized ratio
PBMC=peripheral blood mononuclear cells
DC=dendritic cell
OFA/iLRP=oncofetal antigen/immature laminin receptor protein
mLRP=67 kDa mature laminin receptor protein
Tc=cytotoxic T lymphocyte
Ts=IL-10-secreting, CD8 T cell that inhibits Tc activity IL-10cytokine that can inhibit effector T cell activation and function
moDC=monocyte-derived dendritic cell

Greenberg P A C, Hortobagyi G N, Smith T L, et al. Long-term follow-up of patients with complete remission following combination chemotherapy for metastatic breast cancer. J Clin Oncol. 1996; 14:2197-2205.

Harris J, Morrow M, Norton L. Malignant tumors of the breast. In: DeVita T Jr, Hellman S, Rosenberg S A, eds. Cancer: Principles & Practice of Oncology, 5th ed. Philadelphia, Pa.: Lippincott-Raven Publishers; 1997:15571616.

Chemistry Manufacturing and Control Information Introduction

All ex vivo procedures will be conducted using appropriate GMP standards relevant to phase I clinical studies at the University of South Alabama Department of Microbiology and Immunology using dedicated space and equipment (biosafety cabinets, incubators, etc. in biosafety level II conditions). The space and equipment to be used for the production of the OFA/iLRP-loaded DC vaccine will only be used for that activity. The space used for production of the OFA/iLRP protein and for processing of the patients' peripheral blood mononuclear cells into mature, antigen-loaded dendritic cells comprise 3 physically connected laboratories of between 700 and 1000 square feet approximately. The laboratory in which the OFA/iLRP is produced and purified will not be used in the cell processing and tissue culture. The laboratory space used for cell processing to produce the vaccine contain Class II biological safety cabinets, water-jacketed, CO₂ incubators, a hematology analyzer, and a centrifuge for cell washing, plus other equipment. Cultures will be done in a 95% air/5% CO₂ humidified atmosphere in water-jacketed, CO₂ incubators. The CO₂ level and temperature are internally monitored and maintained by the electronics of the incubator after having been calibrated. Calibrations will be done every month. All of these labs are certified BSL2 laboratories.

General Vaccine Production Protocol

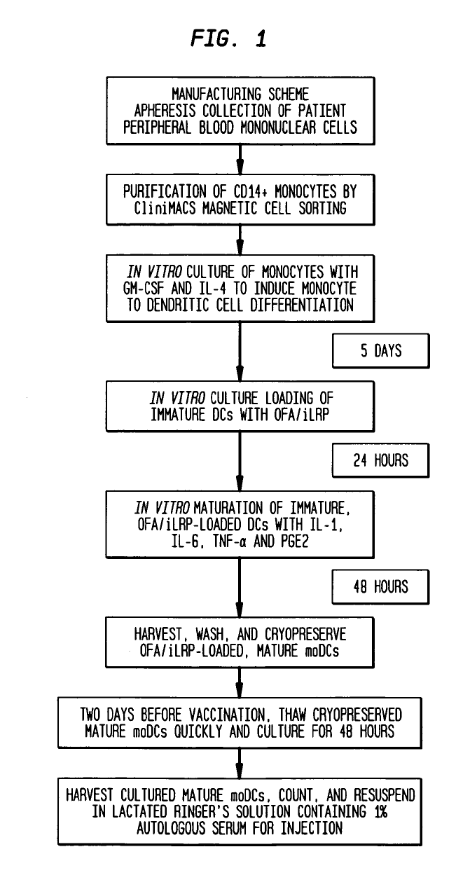

The production process for the proposed DC vaccine involves eight distinct production steps, one relating only to the preparation of purified OFA/iLRP for use as the loading antigen (see below) and seven relating specifically to the isolation, growth, differentiation, maturation, and quantification of the cellular materials from which the mature, antigen-loaded dendritic cells are derived. USA has developed the seven cellular production steps, which are described in greater detail below, include the steps shown in FIG. 1.

As outlined in FIG. 1, to manufacture the OFA/iLRP-loaded, autologous, mature, moDCs, peripheral blood mononuclear cells (PBMC) are collected from the cancer patient by apheresis. Monocytes are purified from the PBMC by anti-CD14-conjugated magnetic microspheres using the CliniMACSPlus instrument by automated, microprocessor-controlled magnetic cell sorting in a closed system. The monocytes are then cultured for 5 days in serum-free medium containing GMP quality GM-CSF and 1L-4 (CellGenix, Antioch, Ill.) to induce their differentiation into immature dendritic cells. The immature moDCs will then be cultured in serum-free medium containing purified OFA/iLRP which the immature moDCs will micropinocytose and become “loaded” with OFA/iLRP. To induce the immature, OFMIRP-loaded moDCs to mature and so become antigen-presenting cells, the OFAALRP-loaded immature moDCs will be cultured for 48 hours in serum-free medium containing GMP quality IL-1, IL-6, TNF-a, and GMP quality prostaglandin E2 (CellGenix, Antioch, Ill.).^{12,52,53,55} The OFA/iLRP loaded, autologous, mature moDCs will then be resuspended in autologous serum containing 5% (v/v) USP glucose and 10% (v/v) DMSO and frozen at −1° C./minute to −80° C. and then transferred into the gas phase of a liquid nitrogen freezer until use.⁶² Two days before immunization, an appropriate number of vials of the cryopreserved OFA/iLRP-loaded, autologous, mature moDCs will be rapidly thawed, resuspended in serum-free medium optimized for DCs and cultured for 48 hours at 37° C. in a humidified 95% air/5% CO₂ atmosphere. A sample of the cells will be counted for viability using Trypan blue dye exclusion and if >70% viable will be pelleted by centrifugation, resuspended in lactated Ringer's solution containing 1% autologous serum and taken up into sterile syringe(s) and transported in an insulated carrier and will be administered to the patient within an hour after delivery. At each of the major production stages before immunization, the product's identity, sterility, and purity will be determined. Those assays will be discussed in subsequent sections below.

Autologous serum to be used in cryopreservation of the antigen-loaded, mature, autologous moDCs and in the vaccine injection solution will be obtained by venipuncture and withdrawal of 50 ml of non-anti-coagulant treated blood from each patient into sterile tubes one week before apheresis purification of the patient's peripheral blood mononuclear cells. The blood will be allowed to sit at room temperature for 30 minutes and then will be refrigerated at 4° C. overnight. The clot that will form will be broken away from the side of the tube (in a Class II biological safety cabinet) with a sterile glass rod. The tube will then be centrifuged at 600×g for 15 minutes at room temperature to pellet the clot and any free blood cells. In a Class II biological safety cabinet, the serum will then be pipetted off of the clot into a sterile tube. A 1 ml sample of the serum will be taken and tested for sterility and endotoxin as described in the section dealing with sterility assays.

During processing of the patients' peripheral blood mononuclear cells to antigen-loaded, mature, autologous moDC, cryopreservation, recovery, culture, and preparation for use in immunization, the cells in blood bags or in cryogenic vials will be labeled with DC/breast carcinoma, the patient's name, date of birth, the batch number of the preparation, date of apheresis, and process stage. The cells will be cultured in sterile Vue-Life Teflon culture bags and centrifuged while remaining in those bags with medium being changed through centrifugation and medium removal using a plasma extractor and a connected waste bag. New media will then be put back into the bag with the cells from a bag of sterile, serum-free DC medium using a sterile tubing connector attached at a tubing connector port on the Vue-Life Teflon culture bag. All processing will be recorded for each patient's cells with date and time and procedure. When the cells are cryopreserved, they will be put in sterile cryogenic vials, labeled with the patient's number, date of apheresis, and process stage and the vials from each patient will go in a separate cryogenic box with the box labeled the same as the tubes. There will be no sharing of cryogenic boxes by tubes containing antigen-loaded, mature, autologous moDC from separate patients. Thus, at all stages, the cells will be in containers with identifying labels that match what is in a book recording the processing events for each patient's cells.

Generation of Mature Antigen-Loaded MoDCs

Step 1—Collection of Patient Peripheral Blood Mononuclear Cells

PBMC will be obtained from the breast cancer patients who have given informed consent through a contract with the American Red Cross cytapheresis unit located in Mobile, Ala. The collection site will follow established procedures under which the Red Cross operates. Dual needle venous access for the cytapheresis procedure will be obtained by the nursing staff at the “Southern Cancer Center.” ACD-A will be used as anticoagulant with an ACD-A:blood ratio of 1:15. Leukapheresis will be performed with a Cobe Spectra Cell Separator (Gambro BCT, Lakewood, Calif.) to process 10-12 L of whole blood with a continuous whole blood inlet flow rate ranging from 50 to 70 ml/min and a collection flow rate of 1 ml/min. The leukapheresis will utilize the MNC standard program on the Cobe Spectra Cell Separator software (version 5.1).⁶¹

While the patient's PBMCs will be sterilely collected into a blood bag under sterile conditions, all other blood components (plasma and other cells) will be returned to the patient during the PBMC purification procedure. After this automated separation, the sterile blood bag containing PBMCs will be disconnected from the cell separator, a 5 ml sample taken to assess viability using Trypan blue dye exclusion counting, sterility testing, and flow cytometric analysis of the cells for expression of certain cell surface markers (see Table IV in the Identity test section below). The bag containing the apheresed PBMCs will be transferred to our cellular immunology BSL-2 containment laboratory for further processing under aseptic conditions. The lenkapheresis expected yield is 15-118×10⁸ viable PBMCs per donor.⁵²⁻⁵³ The minimum acceptable number of PBMCs for this stage will be 10×10⁸ viable PBMCs/donor. Products meeting the minimum cell count, endotoxin and sterility, and cell surface antigen expression specifications will be qualified for further manufacturing.

Step 2—Separation of Monocytes

Monocytes will be purified from the apheresed PBMCs meeting the specifications and qualified for further use (as described above) by positive selection using the GMP quality anti-CD14 antibody-conjugated magnetic bead reagent (Miltenyi Biotec, Auburn, Calif.), the CliniMACS Tubing Set (for up to 20×10⁹ cells), and the CliniMACS Plus automated, microprocessor-controlled magnetic cell sorter instrument (Miltenyi Biotec, Auburn, Calif.) using the method of Campbell et al.,⁵⁴ During separation, the CD14+ cells will be sorted into a sterile, blood bag without ever being exposed to the air. This automated cell sorting process will be done in a Class II biological safety cabinet. The expected yield is 2.25−20×10⁸ viable CD14+ cells (monocytes) at approximately 93-99% purity.⁵³ The minimal acceptable number and purity of monocytes separated by this procedure will be 1.5×10⁸ viable CD14+ monocytes at a purity of 90%. After separation, a 5 ml sample will be obtained to assess viability counting using Trypan blue dye exclusion, sterility testing, and flow cytometric analysis of the cells' expression of certain cell surface markers (see Table IV in the Identity test section, below). The 600 ml bag containing enriched monocytes will be centrifuged at 600×g for 10 minutes at room temperature to concentrate the monocytes. Supernatant will be removed by a Fenwal plasma extractor (Baxter, Deerfield, Ill.) and the pelleted monocytes will be resuspended in Cell-Gro-DC serum-free medium (CellGenix, Antioch, Ill.) and washed again (while remaining in the sterile blood bag) by centrifugation at 600×g for 10 minutes at room temperature. The supernatant will be removed using a Fenwal plasma extractor (Baxter, Deerfield, Ill.) and the cells resuspended to 5 ml in Cell-GroDC serum-free medium, and removed by sterile syringe through a sterile sampling site coupler on the bag. Products meeting the minimum cell count and purity, endotoxin and sterility, and cell surface antigen expression specifications will be qualified as appropriate for further manufacturing.

Step 3—Differentiation of Immature Dendritic Cells

The concentrated monocytes will then be transferred to a sterile Vue-LifeTeflon culture bag (CellGenix, Antioch, Ill.) and enough sterile CellGro DC serum-free medium containing 2500 U/ml of clinical grade GMP quality, sterile human granulocyte/macrophage-colony stimulating factor (GM-CSF) and 1000 U/ml of clinical grade GMP quality, sterile human interleukin-4 (IL-4) (CellGenix, Antioch, Ill.) will be added to the cells in the Vue-Life Teflon culture bag to yield a final concentration of 1×10⁶ viable cells/ml. After aseptically sampling for cell count and sterility testing, the culture bag will be incubated at 37° C. in a humidified 5% CO₂/95% air atmosphere for 5 days. On day 5, the immature monocyte-derived dendritic cells (imoDCs) will be concentrated by centrifugation in the Teflon bags (600×g for 10 minutes at room temperature). The supernatant will be removed using a plasma extractor (Baxter Laboratories, Deerfield, Ill.). A 5 ml sample will be aseptically taken to assess viability by Trypan blue dye exclusion counting, sterility testing, and flow cytometric analysis of immature moDC expression of certain cell surface markers. The major changes that will be assessed is in morphology and increased expression of CD11c. Morphology will be determined both microscopic analysis while doing viability counting and also by doing a quick Wright/Giemsa stain and microscopic counting of the stained cell smears. The expression of CD11c plus the drop in the amount of CD14 expression will be determined by flow cytometric analysis. The cell population must contain minimally 90% immature dendritic cells by morphology and surface marker expression to be loaded with OFA/iLRP.

Step 4—Antigen Loading

New CellGro DC medium containing 100 ng/ml of sterile purified OFA/iLRP will be injected through a sterile sampling site coupler into the sterile VueLife Teflon bags to a final cell concentration of 1×10⁶ cells/ml to 2/3 of the immature moDCs. The bags will be cultured at 37° C. in a humidified 5% CO₂/95% air atmosphere for 24 hours. Following the 24 hour incubation, a 5 ml cell sample will be taken aseptically to assess viability by Trypan blue dye exclusion counting, sterility, and expression of OFA/iLRP by flow cytometric analysis using polyclonal mouse anti-OFA/iLRP IgG antibody and fluorescent anti-mouse IgG antibody. A control staining of some of the cells will be done with polyclonal mouse IgG and fluorescent anti-mouse IgG antibody to make sure staining is not due to non-specific sticking. If >80% of the cells are specifically detectable by polyclonal anti-OFA/iLRP antibody staining and are sterile, they will be induced to become mature antigen-loaded moDCs. Although, the OFA/iLRP is expressed as peptides bound to dendritic cell HLA proteins, the polyclonal anti-OFA/iLRP antibody recognizes multiple epitopes on the OFA/iLRP so that by use of the polyclonal antibody, it will stain the dendritic cells expressing OFA/iLRP peptides on their HLA proteins.

Step 5—Maturation of Antigen-Loaded Dendritic Cells

Subsequently, to mature the antigen-loaded, immature moDCs, a mixture of clinical grade GMP quality, sterile, recombinant human cytokines will be added to the immature moDCs in the Teflon bag to a final cytokine concentration of 10 ng/ml IL-1, 10 ng/ml tumor necrosis factor-α, and 1000 U/mL-6 plus 1.tg/ml prostaglandin E2 (Prostin E2 Sterile Solution, Pharmacia, Ltd, Sandwich, Kent, UK). After aseptic removal of samples for viable cell counting by Trypan blue dye exclusion and sterility test, the bags will be incubated at 37° C. in a humidified 5% CO₂/95% air atmosphere for 48 hours. After 48 hours, the bags containing the mature monocyte-derived dendritic cells (moDCs) will be centrifuged at 600×g for 10 min at room temperature. By the use of a plasma extractor, the supernatant will be removed, and the pelleted mature moDCs will be resuspended in sterile, Cell-Gro-DC serum-free medium and pelleted at 600×g for 10 min at RT. The supernatant will then be removed by plasma extractor, and 20 ml of CellGro DC medium will be added. A 5 ml sample will be taken by syringe for viable cell counting by Trypan blue dye exclusion, sterility testing, and flow cytometric analysis of mature moDC expression of certain cell surface markers (see Table IV in the Identity test section, below). After these procedures, the expected yield is 1−4×10⁹ mature moDCs ⁵². The minimal acceptable number of OFA/iLRP-loaded, autologous, mature moDCs will be 5×10⁸ viable cells. We will also test the mature moDCs for expression of OFA/iLRP by flow cytometric analysis using polyclonal mouse anti-OFA/iLRP IgG antibody and fluorescent anti-mouse IgG antibody to test for presentation of OFA/iLRP peptides on mature moDC HLA proteins as described in step 4 above. By use of a polyclonal anti-OFA/iLRP, which recognizes many OFA/iLRP epitopes, we will be able to detect OFA/iLRP presentation on the mature moDCs. A negative control staining of some of the cells will be done with polyclonal mouse IgG and fluorescent anti-mouse IgG antibody to make sure staining is not due to nonspecific sticking. We must have a minimum of 5×10⁸ viable, OFA/iLRP expressing mature moDCs. Products meeting the minimum cell count, endotoxin and sterility, and cell surface antigen expression specifications will be qualified as appropriate for further manufacturing.

Step 6—Cryopreservation

After viable cell enumeration, cells will be transferred to sterile Nunc cryovials and resuspended in autologous plasma with 5% (v/v) USP glucose and 10% (v/v) DMSO to a final cell concentration of 1×10⁷ mature moDCs/ml in 200 sterile vials. The vials containing the matured antigen-loaded moDC cells will be slowly frozen to −80° C. at a cooling rate of −1°/min. The vials will then be placed in appropriately labeled cryoboxes and the cryoboxes put in the gaseous phase of liquid nitrogen in a liquid nitrogen freezer for cryopreservation. This is done because each patient's OFA/iLRP-loaded mature moDCs will be used for 3 immunizations each one month apart.

Step 7—Reconstitution and Administration of the Vaccine

Two days before immunization, cryogenic vials containing OFA/iLRP pulsed DCs that were matured from that patient's monocytes will be rapidly thawed in a 37° C. water bath, the cells resuspended in 25 ml of CellGro DC serum-free medium, and cultured for 48 hours at 37° C. in a humidified 95% air/5% CO₂ atmosphere in a water-jacketed, CO₂ incubator used only for these DC cultures. After the 48 hour incubation, the cells will be pelleted at 600×g for 10 minutes at room temperature, a sample taken for viability counting by Trypan blue dye exclusion and sterility testing, and pelleted DCs will be resuspended to an appropriate volume of lactated Ringer's solution containing 0.5% autologous plasma to yield 1×10⁷ viable cells/0.05 ml to be used for intradermal injection into the patient. After the liquid nitrogen cryopreservation, we expect to obtain viabilities of >70%, preferably at least about 90%.

We optimize cryopreservation methodology with the apheresis of normal volunteer PBMCs, purification of CD14+ monocytes from the PBMC, induction of monocyte-to-immature DC differentiation, OFA/iLRP loading and ending with induction of maturation of the immature DCs into antigen-loaded, mature moDCs. We will also determine the viability of cryopreserved OFA/iLRP-loaded, mature moDCs prepared from normal volunteers at various times after freezing as well as determining the ability of those moDCs to present OFA/iLRP to and activate autologous T cells specifically. We will also determine the effect of ejecting the prepared cells through 27-30 gauge needles from a syringe on their viability to be sure that the moDCs will survive under the conditions in which they will be injected into the patient. We will also determine if keeping the loaded syringe in an insulated carrier for various times has an effect on the viability of the autologous, mature, OFA/iLRP-loaded moDCs to be used as a vaccine. These extra optimization studies will optimize the methodology such that the best vaccine will be injected into the cancer patients.

### SUMMARY

This sterile closed system employed for this production process, encompassing leukapheresis, CliniMACS selection of monocytes from PBMC and Vue Life Teflon culture bag culturing in CellGro DC medium (CellGenix, Antioch, Ill.) uses the methodology of Mu et al.,⁵² Putz et al.,⁵³ and Campbell et al.,⁵⁴ and the maturation methodology of Mu et al.,⁵² Holt et al.,¹² Rieser et al.,⁵⁵ and Jonuleit et al.⁵⁶ In the generation of dendritic cells from monocytes and the maturation of antigen-loaded, immature dendritic cells only GMP quality, endotoxin-free antibodies and cytokines will be used.

Preparation of Recombinant Human Oncofetal Antigen/Immature Laminin Receptor (rhOFA/iLRP)

The full-length of the human laminin receptor cDNA sequence was amplified by PCR from a plasmid (pDNR-LIB; ATCC) containing the human LBP sequence. A HIS-tag was added at the N-terminal end, followed immediately by a TEV protease site and cloned in a kanamycin-resistent expression vector (pET30; Novagen). The following primers were used to amplify the hOFA cDNA from pDNR-LIB: Forward primer with the restriction enzyme site of NdeI:5′-TCGCATATGCATCACCATCACCATCACGGCTACTGAAAACCATGTATATTCC CAGGGTG-C CATGTC C GGAGC C CTTGATGTC CTGC 3′ (SEQ ID NO.: 1); Reverse primer with the restriction enzyme site of EcoRV:5-ATTTGATATCTTATTAAGACCAGTCAGTGGTTGCTCC-3 (SEQ ID NO.: 2) (Barsuum, et al., Biomaterials 30(17):3091-9 (2009)). PCR parameters were as follows: 98° C. for 10 s, 62° C. for 20 s, 72° C. for 30 s. After the 30th cycle incubation was extended for 5 minutes at 72° C. and held in the PCR machine at 4° C. The sequence of the HIS-tagged OFA/iLRP expression gene was confirmed by extraction of the pET30-h0FA plasmid and sequence analysis of the entire gene (Functional BioSciences, Inc) and found to be identical to published sequence for human LBP from colon carcinoma (GenBank # J03799).

The transformed E. coli strain BL-21 was grown at 37° C. in a broth containing 30 ug/ml kanamycin. Expression of the recombinant protein was induced at ° Dm=1 with 1 mM isopropyl-a-D-thiogalactoside (IPTG). The bacteria were harvested 16 h after induction by centrifugation at 7,000 rpm for 10 minutes.

Bacterial cell lysis and retrieval of inclusion bodies was performed by resuspending the bacterial pellet in CelLytic solution (Sigma-Aldrich; 1 g/10 ml lysis solution) containing lysozyme (2 mg/ml), protease inhibitor cocktail (0.1 ml) and benzonase (500 U). The inclusion bodies were washed three times in 1% 3-[(3-cholamidopropyl) dimethylammonid-1-propanesulfonate in 10 mM Tris-HCl (pH 8.0). This step greatly reduced the level of contaminating LPS. Inclusion body pellets were stored frozen at −70° C. until needed.

Frozen inclusion bodies were thawed and solubilized in 20 volumes of solubilization buffer (10 mM Tris-HCl, 100 mM sodium phosphate, 6 Mguanidine hydrochloride, 10 mM 2-mercaptoethanol, pH 8.0). The solubilized protein solution was clarified by centrifugation (7000 g, 60 rain, 20° C.) and the supernatant fluid was further clarified by filtration (0.2 um). Clarified supernatant was stored at 4° C. prior to chromatographic purification. The purification of the recombinant protein employed a four-step column chromatography process using successively the following matrixes:immobilised metal affinity-, anion exchange-, heparin-, and phenyl-sepharose. A new column was used for each purification run. All buffers used in the purification were sterile filtered using 0.2 pm filters. All chromatography was performed at 20° C.; intermediate fractions were held at 4° C. between each chromatographic step.

The solubilized inclusion bodies were loaded onto a column of Chelating Sepharose FF, precharged with nickel sulfate and equilibrated with 4 column volumes of buffer containing 6 M guanidine, 10 mM Tris-HCl, 100 mM sodium phosphate, pH 8.0. After loading, the column was washed with 6 column volume of 8 M urea, 10 mM Tris-HCl, 100 mM sodium phosphate, pH 8.0 at a flow rate of 2 ml/min. The column was then washed with 10 column volumes of a gradient consisting of 8 M urea, 10 mM Tris-HCl, 100 mM sodium phosphate, pH 8.0 at a flow rate of 2 ml/min, followed by 3 column volumes of 10 mM Tris-HCI, 100 mM sodium phosphate, pH 8.0 before eluting with 2 column volumes of 300 mM imidazole solution in 10 mM Tris-HCl, pH 8.0. Imidazole was then removed by passing the eluted protein on a buffer exchange column equilibrated with 100 mM Tris-HCl buffer, pH 8.

The rOFA was freed from the fusion partner by digestion of the fusion protein with recombinant TEV-protease which has a six-histidine sequence. Intact fusion protein, the TEV-protease and the fragment carrying His-Tag were removed by passing the mixture again through the Nickel chelate column and collecting unbound material.

The rOFA from previous step was purified by chromatography on monoQ column (GE Biosciences). After washing the column with 5 column volumes of the initial buffer, the protein was eluted with a gradient 0-1 M NaCl in 0.05 Tris-buffer, pH 8.0. The active peak containing rOFA was eluted at about 0.5 M NaCl concentration.

The fractions containing the rOFA from the monoQ column were pooled and concentrated by ultrafiltration. The medium was exchanged to 10 mM sodium phosphate (pH 7.0) and loaded on the heparin column. Bound protein was eluted with a linear gradient 0-1 M NaCl in 10 mM sodium phosphate buffer pH 7.0. The fractions containing the rOFA was exchanged into 20 mM bis-Tris buffer, pH 7.0, 1.5 M NaCl and loaded on the phenyl column. Bound protein was eluted with a gradient of 1.5-0 M NaCl and the fractions containing the antigen were combined and the buffer exchanged to PBS and concentrated by ultrafiltration.

Residual endotoxin in rOFA/iLRP was removed by passing the purified material on polymyxin B-agarose column (Detoxi Gel, Pierce). Endotoxin levels were checked by a quantitative chromogenic limulus ainebocyte lysate (LAL) test (BioWittaker, Inc.). Endotoxin levels were <10 endotoxin U/mg protein. After sterile filtration of the antigen (0.2 gm) it was aliquoted into 1 ml sterile vials and frozen (−70° C.) till needed.

Testing of in-Process samples during purification of OFA/iLRP was performed as follows: total protein concentration by Coomassie Blue method; purity by reduced SDS-PAGE; identity be Western blot, using a murine anti-OFA monoclonal antibody (#43515) developed in our laboratory; endotoxin, using a commercial Limulus Amebocyte Lysate testing kit.

Quality Control Testing of Purified OFA/iLRP

The bulk of OFA/iLRP protein solution produced will be tested as follows: sterility, according to US Pharmacopeia methods; endotoxin content using a commercially available kit (Kinetic-QCL 192 Test kit, BioWhittiker, USA); purity by reduced SDS-PAGE; identity by mass spectrometry (MALDITOF-MS); host cell protein content by Western blot analysis using a polyclonal antibody; protein content by amino acid analysis. Stability of the purified, recombinant OFA/iLRP will be determined by reduced SDS-PAGE at various times after production and storage. The OFA/iLRP will be prepared in bulk before any experiments are done so the same lot is used throughout the clinical trial. Before we begin any treatments, the quality control tests listed above will be done.

Description of In-Process and Release Testing for OFA/iLRP-Loaded Dendritic Cells

Sterility

Cell samples will be inoculated into Thioglycolate broth medium to test for contamination of bacteria and fungi using the methods outlined in USP<71>.

The final product will be released based on the results of a Gram stain obtained prior to patient administration. A sample of the final product will also be tested by USP<71>, however results will not be available until 14 days after patient administration. If we find that any of the cells are contaminated before injection, they will be discarded and a new sample obtained by apheresis. Even though we will have Gram stain verification of sterility, full microbiological safety results will not be available at the time of vaccine administration. We will establish an “action plan” for the testing facility to rapidly notify the PI and attending physician in the event the product fails sterility or Mycoplasma testing. The action plan specifies that in the event of such a failure, the patient will be actively monitored for signs of infection. Additionally, any organisms identified by the testing lab will be speciated and tested for sensitivity to antibiotics in the event that treatment of the patient is warranted.

Testing for Mycoplasma Contamination

We will test cell samples for Mycoplasma contamination using PCR methodology that will detect all strains of Mycoplasma (Lamda Biotech, St. Louis, Mo.). We will also utilize the Mycotect culture assay for Mycoplasma contamination (Invitrogen, Carlsbad, Calif.). Samples will be obtained following harvest of mature, antigen-loaded, autologous moDCs, prior to cryopreservation and on the final product. Testing for Mycoplasma will be done on the final product after thawing, but before the final wash. The cells in their cell culture supernatant will be tested for Mycoplasma together at both times in all tests. Since even the rapid PCR test method requires 6-8 hours for results and the Mycotect cell culture method requires 3-4 days, it will be necessary to release the product for patient administration prior to results being available. Consequently, results of Mycoplasma testing of cells in their culture supernatants immediately prior to cryopreservation will be available and used to release the final product.

Purity

PBMCs, purified monocytes, immature DCs, OFA/iLRP-loaded, immature DCs, OFA/iLRP-loaded, mature dendritic cells, and the final product will be tested for the presence of endotoxin using a quantitative chromogenic Limulus amebocyte lysate (LAL) test (Kinetic-QCL 192 Test kit (BioWhittaker, Walkersville, Md.). Samples containing >5 EU/injection will be rejected. The closed apheresis to CliniMACS purification to Vue-life Teflon bag culture system should significantly reduce any contamination especially since we will be working in Class II biosafety cabinets and will be wearing sterile gloves during manipulations.

Identity

TABLE II
Expected Distribution of Differentiation Markers

| | | | Immature | Mature |
|---|---|---|---|---|
| Markers | PBMC | Monocytes | moDC | moDC |
| CD15 | + | + | | − |
| CD14 | + | −H− | ′ | − |
| CD 11c | +/− | +1− | + | −H−H− |
| CD83 | − | − | − | |
| CD80/86 | +/− | +/− | + | i iii |
| Class II MHC | + | + | + | |

Cells will be analyzed by flow cytometry for expression of certain cell surface markers. The apheresed PBMC, purified CD14+ monocytes, immature moDCs, and antigen-loaded, mature moDCs will be stained with flourescentanti-CD15 (pan-myeloid leukocyte protein), anti-CD14 (monocyte-specific), anti-CD11c (expressed more on dendritic cells than monocytes), anti-CD83 (mature dendritic cell-specific), anti-class II MHC (bright on mature dendritic cells), anti-CD80/86 (bright on mature dendritic cells) monoclonal antibodies and analyzed using a FACSVantage flow cytometer (Becton-Dickinson, Mountainview, Calif.). We are using these markers because they will show us both that the differentiation and maturation that should occur under our protocol actually is occurring. The PBMC typically will be 30% lymphocytes which will lower the myeloid marker percentages. The purified CD14+ monocytes should be 90% or more CD14+. The immature moDCs will not have bright CD80/86 fluorescence or bright class II MHC fluorescence, but there should be at least 80% expressing DC markers.⁵⁹ The mature moDCs should be at least 85% bright for class IIMHC,⁵⁷ CD80/86,⁵⁸ and all of the cells that are mature moDCs^{55,56} will express bright CD83 fluorescence in the flow cytometer. These data will show if we are getting the expected cell purification, cell differentiation, and cell maturation intended at each production stage.

To be sure that the autologous, mature moDC are expressing OFA/iLRP after the culture of the immature moDC with the OFA/iLRP, we will conduct flow cytometry with the mature moDC after staining with polyclonal mouse anti OFA/iLRP IgG and fluorescent anti-mouse IgG antibody. For a negative control we will use polyclonal mouse IgG and fluorescent anti-mouse IgG antibody. The polyclonal anti-OFA/iLRP IgG antibody should bind to the OFA/iLRP peptides expressed on the mature moDC class II and class I MHC proteins so the dendritic cells should fluoresce if they took up the OFA/iLRP and processed it appropriately. This is because the polyclonal anti-OFA/iLRP IgG is specific for many OFA/iLRP epitopes so that many peptide epitopes expressed on DC MHC molecules will be able to be bound by the polyclonal anti-OFA/iLRP IgG antibody. For the purpose of release testing >80% of the cells must be positive for presentation of OFA/iLRP peptide.

Viability

A sample of the final formulated cell product will be tested for viability by Trypan blue dye exclusion staining. For the purposes of release testing >70% of the cells must be viable.

Stability

Because we are attempting to make all the OFA/iLRP-loaded, autologous, mature moDCs for a given patient from one apheresis and we will be immunizing 3 times with the OFA/iLRP-loaded, autologous, mature moDCs, we will be cryopreserving the OFA/iLRP-loaded, autologous, mature moDCs. We will determine the viability of the cells subsequent to freezing and thawing and we will also determine the expression of OFA/iLRP and the CD83, class II MHC, and CD80/86 on the thawed cells. We will store the cells in vials and only thaw a vial once. If the cells are not all used from that vial, they will be discarded. Induction of maturation of the OFA/iLRP-loaded, autologous, immature moDCs with TNF-alpha, IL-1, IL-6, and PGE2 should make the cells more able to withstand freezing and thawing plus freezing them in autologous serum containing 5% USP glucose along with 10% DMSO should potentiate the cells' survival. We will not use cells that are less than 70% viable. In our clinical trial, the frozen vials, which will be stored for only three months and thus will not expected to exhibit a significant loss in viability. Nevertheless, we will assess viability, cell density, and purity of each thawed vial and culture after 48 hours (and prior to administration to the patient), as described above. We will also be producing OFA/ILRP-loaded mature moDCs from 5 normal volunteers to be sure our methodology is optimized before beginning work with the cancer patients. We will also determine the effect of freezing and thawing at various times on the ability of the produced OFA/iLRP-loaded, mature, autologous moDCs to specifically activate T cells able to recognize OFA/iLRP from autologous volunteers. Included in that will be cryopreservation and thawing to optimize viability from those cells as well.

We will also determine the stability of the final DC vaccine product under the conditions we are considering for delivery of the vaccine. We will load samples of the DC vaccine in the same type of syringe and needle to used for vaccination and test if the 27-30 gauge needle reduces the DC vaccine viability. We will also load samples of the final DC vaccine product and maintain them under the temperature conditions we are considering for transporting the vaccine to the oncology office. We will test various times at room temperature or on wet ice for changes in vaccine cell viability using Trypan blue dye exclusion.

Efficiency of production of OFA-loaded, autologous, mature monocyte-derived dendritic cell vaccine.

Using the CliniMACS Plus Instrument, to purify the CD14+ monocytes from the peripheral blood mononuclear cells collected by apheresis from 5 L of peripheral blood from the patients, we were able to obtain from 6.1×10⁸ to 22×10⁸ viable CD14+ monocytes. The mean number of CD14+ monocytes purified was 14.75×10⁸ cells. These data are tabulated in Table III below. Upon culture with IL-4, and GM-CSF in clinical GMP grade CellGro DC medium, loading with OFA/iLRP, and induction of maturation with IL-1, IL-6, TNF-alpha, and PGE₂ (all cytokines are clinical GMP quality), we harvested from 0.68×10⁸ to 2.98×10⁸ viable OFA-loaded, mature monocyte-derived dendritic cells. The mean number of DC harvested for the 13 patients is 1.40×108 viable OFA-loaded, mature monocyte-derived dendritic cells/patient. The yield of mature moDCs obtained from the CD14⁺ monocytes put in culture ranged from 3.3 to 21.6% with the mean yield for the 10 patients being 11.09%.

Identity of the purified monocytes and of the mature moDC vaccine.

Tables IV and V below show the results of flow cytometry analysis of appropriate markers to demonstrate that they are the cells that they are supposed to be. Table IV shows the data for the purified monocytes and Table V shows that for the OFA-loaded, mature monocyte-derived DC vaccine. First, the data in Tables IV and V show that the cells are not lymphocytes. Less than 1% of the cells put in the culture or harvested out of the culture express CD3 or CD19 and the fluorescence intensity seen is background fluorescence levels. CD3 and CD19 are markers of T and B lymphocytes, respectively. Monocytes express CD14, some CD86, and some HLA-DR, but no CCR7. Monocytes and dendritic cells (DC) express CD11c (because it is on all monocytes, macrophages, and monocyte-derived dendritic cells). Mature DCs do not express CD14, express more CD86 than monocytes, express high levels of HLA-DR, and express CCR7. Only mature DCs express the protein CD83.

The data in Table IV demonstrate that the CD14+ cells purified from the patients' apheresis collected peripheral blood mononuclear cells show the markers of monocytes. The data shown in Table V demonstrate that our vaccine DCs express high levels of CD86, HLA-DR and express CCR7. It also shows that the vaccine DCs are CD83+ and so are mature DCs. The increased expression of CD86 and HLA-DR by mature DCs is one reason why they are so good at activating T cells. The increased HLA-DR shows that major histocompatibility complex (MHC) proteins are expressed at high levels and it is on those that the peptides are presented to T cell antigen receptors.

The increased amount of CD86 on mature monocyte-derived DCs allows for increased activation of antigen-recognizing T cells because that protein is bound by a T cell surface protein to complete T cell activation. Therefore, the more MHC proteins and CD86 that is expressed by a DC, the better it will be at activating T cells that can recognize the antigenic peptides presented by the DC. The expression of CCR7 (a receptor for a chemotactic protein that is produced by stromal cells in lymph nodes) allows the mature DCs to migrate to the lymph nodes. Therefore, our DC vaccine when injected will migrate to the lymph nodes to present the OFA/iLRP peptides presented on the DC MHC proteins to T cells that migrate into the lymph nodes. This will facilitate activation of anti-OFA/iLRP T cell immune responses. The CliniMACS Plus Instrument-purified CD14⁺ cells (Table IV) were from 92.8% CD14⁺ to 99.9% CD14⁺ (mean=98.4% with a mean MFI of 24,590). These cells were also CD11c⁺, CD86+, and HLA-DR⁺, but did not express CD83, CCR7, nor CD3 or CD19, which one would expect for monocytes. While >80% of the purified monocytes expressed CD86 and HLA-DR they were considerably less bright than was seen with the mature moDCs which would be expected because as DCs mature, they produce much more MHC protein (as represented by HLA-DR) and CD86 in order to be able to efficiently interact and activate T cells that recognize the antigenic peptides the DCs are presenting. Microscopic examination of the purified monocytes obtained with the CliniMACS Plus Instrument showed a uniform morphology consistent with being monocytes and were >90% viable when counted before being placed in culture to generate DCs.

Table V shows the flow cytometry data of the markers on the harvested OFA-loaded, autologous, mature, monocyte-derived dendritic cells. The mature moDCs expressed CD86 (mean MFI=2744), high levels of HLA-DR (mean MFI=44,780), CCR7 (mean MFI=1567) and CD83 (mean MFI=2707), but only isotype control levels of CD14 (mean MFI=760). The data in Table V shows that the mean percentage of vaccine cells expressing CD83, CD86, and HLA-DR, respectively were 96.7%, 93.4%, and 99.4% while the mean percentage of vaccine cells expressing CD14, CD3, and CD19, respectively were 0.5%, 0.6%, and 0.5%. Thus, the vaccine cells we produce meet the quality control levels we set for the FDA. Also, it means that the DCs harvested do not express CD14 (a monocyte marker) nor CD3 or CD19 (lymphocyte markers) above background fluorescence levels. That the harvested DCs used for the vaccine express CD83, high levels of CD86 and HLA-DR show that these cells are mature DCs. Also, the expression of CCR7 by the harvested DCs means these cells should migrate to the lymph nodes draining the site of injection when injected intradermally into the patient's skin. That will facilitate the activation of OFA/iLRP-specific T cells in the patient. All vaccine preps were >88% viable even after being stored frozen in liquid nitrogen for 3 months and then thawed and cultured for 48 hours before being used for the third immunization of the patient.

Table VI shows that the vaccine preps all had acceptably low levels of endotoxin and were all found to be sterile using the culture method detailed in USP chapter 71 on sterility tests. Also, PCR assay of samples of the harvested vaccine cells and culture medium were all negative for Mycoplasma contamination. Thus, we were able to produce sufficient numbers of vaccine cells for immunization and for quality control assays plus the vaccine cells expressed the appropriate cell surface markers and the vaccine was sterile with low levels of endotoxin.

TABLE III
Monocyte Enrichment and Mature MoDC Generation
from PBMC of Breast Cancer Patients

| | | | | Mature | Mature |
|---|---|---|---|---|---|
| | | CD14+ | CD14+ | OFA- | MoDC |
| | | Cells | Cells | loaded | yield (% |
| | PBMC | (monocytes) | (% of | MoDC | of CD14+ |
| Patients | (×10⁸) | (×10⁸) | PBMC) | (×10⁸) | Cells) |
| 1006 | 17.82 | 6.1 | 34.2 | 1.26 | 20.7 |
| 1008 | 125.08 | 13.8 | 11.0 | 2.98 | 21.6 |
| 1010 | 100.11 | 15.7 | 15.7 | 2.40 | 15.3 |
| 1011 | 82.16 | 16.8 | 20.4 | 0.78 | 4.6 |
| 1013 | 205.44 | 9.1 | 4.5 | 1.97 | 21.6 |
| 1014 | 116.16 | 20.5 | 17.1 | 0.68 | 3.3 |
| 1015 | 156.17 | 22.0 | 14.1 | 1.16 | 5.3 |
| 1016 | 72.00 | 7.4 | 10.3 | 0.96 | 13.0 |
| 1017 | 124.74 | 14.9 | 11.9 | 1.10 | 7.4 |
| 1018 | 85.36 | 19.9 | 23.3 | 0.83 | 4.2 |
| 1019 | 141 | 12.9 | 9.1 | 1.52 | 11.8 |
| 1020 | 161 | 21.7 | 13.5 | 1.72 | 8.0 |
| 1021 | 48.11 | 10.9 | 22.6 | 0.80 | 7.4 |
| Mean | 110.40 | 14.75 | 15.98 | 1.40 | 11.09 |
| SD | 50.4 | 5.4 | 7.71 | 0.7 | 6.81 |

TABLE IV
Flow Cytometry Analysis of Surface Antigen Phenotype Monocytes

| | CD11c | CD14 | CD83 | CD86 | HLA-DR | CCR7 | CD3 | CD19 |
|---|---|---|---|---|---|---|---|---|
| Patients | %+/MFI | %+/MFI | %+/MFI | %+/MFI | %+/MFI | %+/MFI | %+/MFI | %+/MFI |
| 1006 | 85.9/1127 | 98.4/12309 | 0.2/195 | 92.1/1031 | 91.5/1686 | 0.5/586 | 0.5/487 | 0.4/492 |
| 1008 | 88.9/1188 | 99.4/13598 | 0.3/223 | 93.5/1075 | 87.6/1937 | 0.4/617 | 0.4/523 | 0.6/583 |
| 1010 | 86.9/1809 | 99.3/26694 | 0.2/443 | 88.2/1086 | 93.9/2853 | 0.3/462 | 0.6/567 | 0.7/527 |
| 1011 | 90.6/1914 | 99.4/15155 | 0.5/448 | 86.1/1445 | 93.5/3654 | 0.4/527 | 0.5/492 | 0.4/462 |
| 1013 | 93.8/1244 | 99.8/69032 | 0.4/513 | 89.4/1963 | 86.5/991 | 0.5/450 | 0.4/585 | 0.3/533 |
| 1014 | 94.6/1522 | 99.3/32211 | 0.3/732 | 89.9/1362 | 91.0/2486 | 0.3/422 | 0.7/601 | 0.5/559 |
| 1015 | 99.8/2883 | 99.9/25374 | 0.6/417 | 99.4/1184 | 88.5/2773 | 0.4/535 | 0.3/537 | 0.4/497 |
| 1016 | 90.6/1250 | 98.0/9842 | 0.8/317 | 89.5/796 | 86.7/1564 | 0.3/326 | 0.5/622 | 0.5/528 |
| 1017 | 94.6/2121 | 97.5/8689 | 0.2/273 | 92.1/864 | 93.4/4481 | 0.9/646 | 0.6/549 | 0.5/493 |
| 1018 | 87.4/2325 | 98.9/23390 | 0.4/336 | 87.4/1482 | 91.2/2136 | 0.3/545 | 0.4/701 | 0.5/532 |
| 1019 | 87.9/943 | 99.8/69690 | 0.2/267 | 87.6/954 | 93.3/1859 | 0.8/996 | 0.6/654 | 0.3/622 |
| 1020 | 85.1/899 | 96.8/9931 | 0.4/763 | 84.1/754 | 89.0/1048 | 0.9/959 | 0.7/436 | 0.6/517 |
| 1021 | 81.2/1210 | 92.8/3757 | 0.9/1434 | 80.6/1745 | 83.7/1464 | 0.1/822 | 0.3/544 | 0.4/612 |
| Mean | 89.9/1572 | 98.4/24590 | 0.4/489 | 89.2/1198 | 90.0/1226 | 0.5/607 | 0.5/561 | 0.5/534 |

TABLE V
Flow Cytometry Analysis of Surface Antigen Phenotype OFA-loaded Mature MoDC

| | CD11c | CD14 | CD83 | CD86 | HLA-DR | CCR7 | CD3 | CD19 |
|---|---|---|---|---|---|---|---|---|
| Patients | %+/MFI | %+/MFI | %+/MFI | %+/MFI | %+/MFI | %+/MFI | %+/MFI | %+/MFI |
| 1006 | 86.2/1421 | 0.5/573 | 98.3/2247 | 96.7/3244 | 98.9/26636 | 94.1/1237 | 0.6/554 | 0.4/484 |
| 1008 | 90.1/1334 | 0.2/327 | 96.6/1548 | 97.4/1967 | 97.9/37777 | 93.7/1183 | 0.7/566 | 0.5/528 |
| 1010 | 86.7/1256 | 0.6/811 | 97.1/2232 | 93.9/2817 | 99.5/29382 | 95.2/1673 | 0.4/603 | 0.3/641 |
| 1011 | 88.1/3036 | 0.3/1071 | 95.4/2831 | 97.4/4546 | 99.3/27720 | 90.4/1385 | 0.5/611 | 0.5/599 |
| 1013 | 86.2/2122 | 0.4/625 | 97.5/2074 | 92.3/2745 | 99.8/80535 | 99.3/1577 | 0.7/527 | 0.5/583 |
| 1014 | 90.3/1356 | 0.7/943 | 96.8/2948 | 92.7/2267 | 99.6/18672 | 90.3/1117 | 0.6/588 | 0.5/527 |
| 1015 | 99.2/2192 | 0.5/397 | 97.4/2457 | 98.9/2388 | 99.7/23397 | 92.4/1413 | 0.5/489 | 0.6/513 |
| 1016 | 96.2/873 | 0.3/1088 | 95.7/1435 | 92.9/1402 | 99.6/25478 | 99.5/2489 | 0.6/591 | 0.5/612 |
| 1017 | 89.4/1236 | 0.6/858 | 99.2/8491 | 91.9/3337 | 99.7/32386 | 99.3/1846 | 0.4/567 | 0.4/618 |
| 1018 | 90.6/1574 | 0.4/641 | 99.2/1148 | 91.8/2360 | 99.1/27587 | 90.6/1771 | 0.5/632 | 0.4/588 |
| 1019 | 87.3/1626 | 0.5/646 | 87.6/2068 | 86.7/2839 | 99.8/62317 | 86.2/1604 | 0.7/486 | 0.6/588 |
| 1020 | 86.7/1898 | 0.8/1135 | 99.9/3004 | 88.4/2013 | 100/145471 | 85.1/1513 | 0.8/883 | 0.7/697 |
| 1021 | Not | Not | Not | Not | Not | Not | Not | Not |
| | done | done | done | done | done | done | done | done |
| Mean | 90.0/1660 | 0.5/760 | 96.7/2707 | 93.4/2744 | 99.4/44780 | 93.0/1567 | 0.6/591 | 0.5/582 |

TABLE VI
Purity and Sterility of OFA-loaded
Mature MoDCs

| | Endotoxin | | Mycoplasma- |
|---|---|---|---|
| Patients | (EU/ml) | Sterile | free (by PCR) |
| 1006 | 1.4937 | Yes | Yes |
| 1008 | 1.4194 | Yes | Yes |
| 1010 | 1.4643 | Yes | Yes |
| 1011 | 0.9725 | Yes | Yes |
| 1013 | 0.0677 | Yes | Yes |
| 1014 | 0.1059 | Yes | Yes |
| 1015 | 0.0590 | Yes | Yes |
| 1016 | 0.2875 | Yes | Yes |
| 1017 | 0.0496 | Yes | Yes |
| 1018 | 2.6230 | Yes | Yes |
| 1019 | 3.3436 | Yes | Yes |
| 1020 | 0.0229 | Yes | Yes |
| Mean | 0.9924 | | |

Measurement of Anti-OFA/iLRP T Cell and Antibody Responses in Vaccinated Breast Cancer Patients

As described below, the relative frequencies of interferon-gamma secreting T cells, Il-10 secreting T cells, and optionally Il-4 secreting T cells may be determined by isolating or purifying CD4 and CD8 T cells from the patient's blood and then culturing the cells with OFA/iLRP-loaded autologous monocytes (isolated or purified from the same patient, preferably from the same blood sample) and then placing 100,000 T cells into ELISPOT plates with the wells coated with anti-IL-10, anti-IL-4, or anti-gamma interferon antibody, to capture/bind the cytokines secreted. When the cells are washed away after incubation, and then after the plates are washed and a biotin-conjugated, second anti-cytokine antibody is introduced, followed by incubation, washing the plates, and addition of enzyme-conjugated avidin, incubation, washing the plates, then addition of substrate and incubation, and at a set time stopping the reaction. Cells that secret the appropriate cytokine captured by the plate-bound antibodies are easily identified visually by the presence of colored circles. By counting the circles and comparing to controls, the frequency of CD4 or CD8 gamma interferon-secreting T cells (Th1 and Tc cells), the frequency of CD4 IL-4-secreting T cells (Th2 cells), and the frequency of IL-10-secreting CD4 and CD8 T cells (Treg and Is cells) are determined.

More specifically, determination of frequency of IFN-y, IL-4, and IL-10-secreting T cells in PBMC of patients using ELISPOT assays OFA/iLRP is an immunoregulatory molecule capable of inducing both TM and Th2 cytokine responses (e.g., IFN-y, IL-4 and IL-10). On the day of the first immunization injection and at 2 month intervals, thereafter, 10 ml of heparinized blood will be drawn and T lymphocytes purified by negative selection using the Rosette-Sep methodology as described by Stem Cell Technologies (Vancouver, BC).This involves adding Rosette-Sep tetrameric antibody reagent for T cell purification to the heparinized blood, incubating at room temperature for 20 minutes and then layering the blood cells over Ficoll-Paque Plus and centrifuging at 1200×g for 20 minutes at room temperature (with no brake). The T lymphocytes will be at the plasma/Ficoll-Paque Plus interface and all other blood cells will have been aggregated and be pelleted at the bottom of the centrifuge tube. This is because the Rosette-Sep antibody reagent for T cell purification includes antibodies which bind glycophorin on RBC and markers on the non-T lymphocyte cells of the blood so that all become bound to RBC and so aggregate and pellet while T lymphocytes layer where PBMC normally reside. Thus, the T lymphocytes are purified by negative selection using this method and normally contain >97% T lymphocytes. The purified T cells will be washed, resuspended in complete Megacell RPMI-1640 medium (Sigma Chemical Co., St. Louis, Mo.) and a viability count done using Trypan blue dye exclusion. A sample of the cells will be analyzed by flow cytometry for CD3 expression. Thawed, medium washed autologous OFA/iLRP- or ovalbumin-pulsed mature moDCs produced during ex vivo production of the vaccine will serve as antigen-presenting cells and so will be x-irradiated with 3000 rad and then kept on ice until the proliferation and ELISPOT cultures will be set up.

The T lymphocytes will be separated into CD4+ and CD8+ subsets by negative selection of the purified T cells with anti-CD8 and anti-CD4 monoclonal antibody-coated magnetic beads, respectively, using the IMag magnet (BD, San Diego, Calif.). Removal of the medium and cells not bound by the magnet subsequent to anti-CD8 and anti-CD4 mAb-coated magnetic bead treatment and exposure to the magnet will yield the CD4+ and CD8+ T cells. The cells will then be washed in medium by centrifugation, resuspended in complete Megacell RPMI-1640 medium and viability counts done. Purity of the fractionated subsets will be determined on a sample of the purified cells by flow cytometry using fluorescent-tagged anti-CD3, anti-CD4 and anti-CD8 antibodies.

ELISPOT Assay

In order to determine the types of T lymphocytes induced by the dendritic cell immunization, we will use an ELISPOT assay and analyze it with the Immunospot analyzer (Cellular Technology, Cleveland). To do this, we will set up essentially the same assay as above (Purified patient CD4 or CD8 T cells will be titrated into ELISPOT plate wells containing optimal numbers of irradiated autologous, OFA/iLRP- or ovalbumin-pulsed mature moDCs to be sure we have the optimal number of spots/well. These cultures will be set up in ELISPOT plates coated with either monoclonal anti-IL-4, IL-10, or anti-γ-interferon capture antibodies. The cells will be cultured for 24 hours and then the cells carefully washed off the plates. Biotinylated detection antibodies (a different monoclonal anti-IL-4, anti-IL-10, or anti-γ-interferon antibody) will be added, the plates incubated at 4° C. for 12 hours and then each well will be washed to remove unbound antibody. This is followed by a 2-hour incubation with either streptavidin (SA)-horseradish peroxidase. The 3-amino-9-ethylcarbazole substrate will be added, incubated at room temperature for 5-30 minutes, the reaction stopped and the red spots resulting from cytokine secretion and being bound to the membrane bottom of the ELISPOT well will be analyzed with the immunospot analyzer customized for analyzing ELISPOTs to meet objective criteria for size, chromatic density, shape, and color. Significant increase over baseline will be evaluated statistically. Statistical significance of results will be evaluated by student's t test for unpaired samples. Values of P<0.05 will be considered as significant. By combining the proliferation assay data with the ELISPOT data we will know the frequency of OFA/iLRP-specific CD8 T cells secreting γ-interferon (cytotoxic T cells),^{17,50} the frequency of OFA/iLRP-specific CD8 T cells secreting IL-10 (Ts cells),^{17,51} as well as the frequency of OFA/iLRP specific Th1 (CD4, gamma interferon-secreting) and Th2 (CD4, IL-4 and IL-10-secreting) T cells. These data will be useful in interpreting the clinical results of OFA/iLRP-pulsed moDC immunization of breast cancer patients. It may allow an explanation of why certain patients may show little or no relief from the tumor after receiving the immunization. Also, by performing the analyses of peripheral blood T cells at the various times mentioned above, the effect of both the cancer and the immunizations can be seen in the anti-OFA/iLRP T cell immune responses as time progresses. The investigators performing the proliferation and ELISPOT analyses, however, will not know the clinical status of the patients so that the scoring cannot be tainted by bias.

The ELISPOT analysis of cytokine-secreting, OFA/iLRP-specific CD4 and CD8 T cells described above, the OFA/iLRP-induced proliferation assay described below, and the enumeration of OFA/iLRP-specific CD4 and CD8 T regulatory cells (described below) will also be done with T cells purified from the patients' blood at the beginning of the study (before any immunization injections) and from control, non-treated normal volunteers in order to more clearly determine the effects of the immunizations and the cancer on the T cell immunity. This will also allow us to see what kind of OFA/ILRP-specific T cell immunity exists in normal humans as well as in the breast cancer patients before immunization.¹⁷

Screening Patients' Sera for anti-OFA/iLRP antibodies by ELISA

Serum Ab binding to rOFA will be assessed by ELISA. rOFA in PBS (5 μg/ml) will be used at 1000/well to a 96-well polystyrene ELISA plates (Dynatech Laboratories). Ovalbumin at the same concentration will be used as the negative control protein. The plates will be incubated at 4° C. overnight, washed with PBS containing 0.05% Tween 20 (PBS-T) and blocked for 60 min with PBS-T containing 2% BSA (PBS-TB). After washing, patients and healthy control sera (starting dilution of 1/100) diluted in PBS-TB will be transferred to the plates and incubated for 2 hours at room temperature. The plates will then be washed, and biotinylated goat anti-human IgG (γ-chain-specific) will be added at a dilution of 1/4000. For IgM Ab, biotinylated goat anti-human IgM (p-chain-specific) will be added at the same dilution. Following a 60-min incubation, the plates will be washed and ABC reagent (biotinylated horseradish peroxidase: avidin; Vector Laboratories, Inc.) added. The plates will then be washed and substrate solution containing ABTS reagent (Zymed) will be added in 0.1 M citrate buffer, pH 4.0, and 0.015% H₂O₂. After incubation for 30 min, plates will be read on a microtiter plate reader at OD405. Sera will be considered positive when the absorbance value exceeds the mean absorbance of the control plus three times SD at the same dilution.

Accordingly, the antibodies so identified and isolated are within the invention.

The invention also provides antibodies recognizing specific epitopes of OFA/iLRP. Methods for producing antibodies are well known in the art. In general, a protein or a fragment thereof can be used as an immunogen to generate antibodies using standard techniques for polyclonal and monoclonal antibody preparation. Typically, an antigenic peptide comprises at least 8 amino acid residues. An immunogen is used to prepare antibodies by immunizing a suitable subject (e.g., rabbit, goat, mouse, or other mammal) with the immunogen. The preparation can further include an adjuvant, such as Freund's complete or incomplete adjuvant, or similar immunostimulatory agent. Immunization of a suitable subject with an immunogenic preparation induces a polyclonal antibody response. The antibody titer in the immunized subject can be monitored over time by standard techniques, such as an enzyme linked immunosorbent assay (ELISA) using immobilized antigen. If desired, the antibody molecules directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77 96), ortrioma techniques. Alternative, a monoclonal antibody can be identified and isolated by screening a recombinant combinatorial immunoglobulin library (e.g., an antibody phage display library) with an antigen to thereby isolate immunoglobulin library members that bind to the antigen. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example, using methods described in Better et al. (1988) Science 240:1041-1043; Liu et al. (1987) Proc. Natl. Acad. Sci. USA 84:3439-3443; Nishimura et al. (1987) Canc. Res. 47:999-1005.

### LIST OF REFERENCES

1. Bitton R J. Cancer vaccines: a critical review on clinical impact. Curr Opin Mal Ther. 2004; 6:1726.
2. O'Shaughnessy J A. Breast Cancer Vaccines. Clin Breast Cancer. 2003; 3:5133.
3. Romani N, Reider D, Heuer M et al. Generation of mature dendritic cells from human blood. An improved method with special regard to clinical applicability. J Immunol Methods. 1996; 196:137-151.
4. Bender A, Sapp M, Feldman M et al. Dendritic cells as immunogens for human CTL responses. Adv Exp Med. Biol. 1997; 417:383-387.
5. Bender A, Sapp M, Schuler G, Steinman R M, Bhardwaj N. Improved methods for the generation of dendritic cells from nonproliferating progenitors in human blood. J Immunol Methods. 1996; 196:121-135.
6. Schreurs M W, de Boer A J, Figdor C G, Adema G J. Genetic vaccination against the melanocyte lineage-specific antigen gp100 induces cytotoxic T lymphocyte-mediated tumor protection. Cancer Res. 1998; 58:2509-2514.
7. Gilboa E, Nair S K, Lyerly H K. Immunotherapy of cancer with dendritic-cell-based vaccines. Cancer Immunol Immunother. 1998; 46:82-87.
8. Nair S K, Hull S, Coleman D et al. Induction of carcinoembryonic antigen (CEA)-specific cytotoxic T-lymphocyte responses in vitro using autologous dendritic cells loaded with CEA peptide or CEA RNA in patients with metastatic malignancies expressing CEA. Int J. Cancer. 1999; 82:121-124.
9. Banchereau J, Palucka A K, Dhodapkar M et al. Immune and clinical responses in patients with metastatic melanoma to CD34(+) progenitor-derived dendritic cell vaccine. Cancer Res. 200161-6451-6458.
10. Palucka A K, Dhodapkar M V, Paczesny S et al. Single injection of CD34+ progenitor-derived dendritic cell vaccine can lead to induction of T-cell immunity in patients with stage 1V melanoma. J. Immunother. 2003; 26:432-439.
11. Schuler G, Schuler-Thurner B, Steinman R M. The use of dendritic cells in cancer immunotherapy. Curr. Opin. Immunol. 2003; 15:138-147.
12. Holt, L, Zelle-Rieser C, Gander H et al. Immunotherapy of metastatic renal cell carcinoma with tumor lysate-pulsed autologous dendritic cells. Clin Cancer Res. 2002; 8:3369-3376.
13. Nestle F O, Alijagic 5, Gilliet M et al. Vaccination of melanoma patients with peptide- or tumor lysate-pulsed dendritic cells. Nat. Med. 20 1998; 4:328-332.
14. Coulie P G, Karanikas V, Lurquin C et al. Cytolytic T-cell responses of cancer patients vaccinated with a MAGE antigen. Immunol Rev. 2002; 188:33-42.
15. Svane I M, Pedersen A E, Johnsen H E et al. Vaccination with p53peptide-pulsed dendritic cells, of patients with advanced breast cancer: report from a phase I study. Cancer Immunol Immunother. 2004; 53:633-641.
16. Figdor C G, de Vries I J, Lesterhuis W J, Melief C J. Dendritic cell immunotherapy: mapping the way. Nat. Med. 2004; 10:475-480.
17. Rohrer J W, Barsoum A L, Dyess D L, Tucker J A, Coggin J H, Jr. Human breast carcinoma patients develop clonable oncofetal antigen-specific effector and regulatory T lymphocytes. J. Immunol. 1999; 162:6880-6892.
18. Zelle-Rieser C, Barsoum A L, Sallusto F et al. Expression and immunogenicity of oncofetal antigen-immature laminin receptor in human renal cell carcinoma. J. Urol. 2001; 165:1705-1709.
19. Coggin J H, Jr., Barsoum Ala., Rohrer J W. 37 kilo Dalton oncofetal antigen protein and immature laminin receptor protein are identical, universal T cell inducing immunogens on primary rodent and human cancers. Anticancer Res. 1999; 19:5535-5542.
20. Coggin J H, Jr. Classification of tumor-associated antigens in rodents and humans. Immunol Today. 1994; 15:246-247.
21. Barsoum A L, Coggin J H, Jr. Isolation and partial characterization of a soluble oncofetal antigen from murine and human amniotic fluids. Int J. Cancer. 1991; 48:248-252
22. Payne W J, Jr., Coggin J H, Jr. Mouse monoclonal antibody to embryonic antigen: development, cross-reactivity with rodent and human tumors, and preliminary polypeptide characterization. J Nati Cancer Inst. 1985; 75:527-544.
23. Barsoum A L, Coggin J H, Jr. Immunogenicity of a soluble partially purified oncofetal antigen from murine fibrosarcoma in syngeneic mice. J Biol Response Mod. 1989; 8:579-592.
24. Wewer U M, Liotta L A, Jaye M et al. Altered levels of laminin receptor mRNA in various human carcinoma cells that have different abilities to bind laminin. Proc Nati Acad Sci USA. 1986; 83:7137-7141.
25. Coggin J H, Jr., Barsoum. A L, Rohrer J W. Tumors express both unique TSTA and crossprotective 44 kDa oncofetal antigen. Immunol Today. 1998; 19:405-408.
26. Siegel S, Wagner A, Kabelitz D et al. Induction of cytotoxic T-cell responses against the oncofetal antigen-immature laminin receptor for the treatment of hematologic malignancies. Blood. 2003; 102:4416-4423.
27. Gussack G S, Rohrer S D, Hester R B, Liu P I, Coggin J H, Jr. Human squamous cell carcinoma lines express oncofetal 44-kD polypeptide defined by monoclonal antibody to mouse fetus. Cancer. 1988; 62:283-290.
28. Menard 5, Castronovo V, Tagliabue E, Sobel M E. New insights into the metastasis-associated 67 kD laminin receptor. J Cell Biochem. 1997; 67155165.
29. Castronovo V. Laminin receptors and laminin-binding proteins during tumor invasion and metastasis. Invasion Metastasis. 1993; 13:1-30.
30. Coggin J H, Jr., Rohrer S D, Hester R D et al. 44-kd oncofetal transplantation antigen in rodent and human fetal cells. Implications of recrudescence in human and rodent cancers. Arch Otolaryngol Head Neck Surg. 1993; 119:1257-1266.
31. Coggin J H, Jr. Oncofetal antigens. Nature. 1986; 319:428.
32. Coggin J H, Jr., Rohrer J W, Barsoum A L. True immunogenicity of oncofetal antigen/immature laminin receptor protein. Cancer Res. 2004; 64:4685.
33. Tjoa B A, Simmons S J, Bowes V A et al. Evaluation of phase I/II clinical trials in prostate cancer with dendritic cells and PSMA peptides. Prostate 1998; 36:39-44.
34. Heiser A, Coleman D, Dannull J et al. Autologous dendritic cells transfected with prostate-specific antigen RNA stimulate CTL responses against metastatic prostate tumors. J. Clin. Invest. 2002; 109:409-417.
35. Vonderheide R H, Domchek S M, Schultze J L et al. Vaccination of cancer patients against telomerase induces functional antitumor CD8+ T lymphocytes. Clin. Cancer Res. 2004; 10:828-839.
36. Mu L J, Kyte J A, Kvalheim G et al. Immunotherapy with allotumor mRNA-transfected dendritic cells in androgen-resistant prostate cancer patients. Br. J. Cancer 2005; 93:749-756.
37. Schuler-Thurner B, Schultz E S, Berger T G et al. Rapid induction of tumor-specific type I helper cells in metastatic melanoma patients by vaccination with mature, cryopreserved, peptide-loaded, monocyte-derived dendritic cells. J. Exp. Med. 2002; 195:1279-1288.
38. de Vries I J M, Lesterhuis W J, Scharenborg N M et al. Maturation of dendritic cells is a prerequisite for inducing immune responses in advanced melanoma patients. Clin. Cancer Res. 2003; 9:5091-5100.
39. Jonuleit H, Giesecke-Tuettenberg A, Tuting T et al. A comparison of two types of dendritic cell as adjuvants for the induction of melanoma-specific T cell responses in humans following intranodal injection. Int. J. Cancer 2001; 93:243-251.
40. Hernando J J, Park T-W, Fischer, H-P et al. Vaccination with dendritic cells transfected with mRNA-encoded folate-receptor-a for relapsed metastatic ovarian cancer. Oncology. The Lancet.com 2007; 8:451-454.
41. Inaba K, Inaba M, Romani N et al. Generation of large numbers of dendritic cells from mouse bone marrow cultures supplemented with granulocyte/macrophage colony stimulating factor. J. Exp. Med. 1992; 176:1693-1702.
42. Rohrer J W, Barsoum A L, Coggin J H, Jr. Identification of oncofetal antigen/immature laminin receptor protein epitopes that activate BALB/c mouse OFAhLRP-specific effector and regulatory T cell clones. J. Immunol. 2006; 176:2844-2856.
43. Morse M A, Coleman R E, Akabani G et al. Migration of human dendritic cells after injection in patients with metastatic malignancies. Cancer Res. 1999; 59:56-58.
44. Fong L, Brockstedt D, Benike C, Wu L, Engleman E G. Dendritic cells injected via different routes induce immunity in cancer patients. J. Immunol. 2001; 166:4254-4259.
45. Fong L, Brockstedt D, Benike C et al. Dendritic cell-based xenoantigen vaccination for prostate cancer immunotherapy. J. Immunol. 2001; 167:7150-7156.
46. Slingluff C L, Jr., Petroni G R, Yamshchikov G V et al. Clinical and immunologic results of a randomized phase II trial of vaccination using four melanoma peptides either administered in granulocyte-macrophage colony-stimulating factor in adjuvant or pulsed on dendritic cells. J Clin Oncol. 2003; 21:4016-4026.
47. Bedrosian I, Mick R, Xu S et al. Intranodal administration of peptide-pulsed mature dendritic cell vaccines results in superior CD8+ T-cell function in melanoma patients. J Clin Oncol. 2003; 21:3826-3835.
48. Banchereau J, Paczesny S, Blanco P et al. Dendritic cells: controllers of the immune system and a new promise for immunotherapy. Ann N Y Acad. Sci. 2003; 987:180-187.
49. Helms T, Boehm B O, Asaad R J et al. Direct visualization of cytokine-producing recall antigen-specific CD4 memory T cells in healthy individuals and HIV patients. J. Immunol. 2000; 164:3723-3732.
50. Neidhardt-Berard E M, Berard F, Banchereau J, Palucka A K. Dendritic cells loaded with killed breast cancer cells induce differentiation of tumor-specific cytotoxic T lymphocytes. Breast Cancer Res. 2004; 6:R322-R328.
51. Martin-Fontecha A, Sebastiani S, Hopken U E et al. Regulation of dendritic cell migration to the draining lymph node: impact on T lymphocyte traffic and priming. J Exp Med. 2003; 198:615-621.
52. Mu L J, Gaudernack G, Saeboe-Larssen S et al. A protocol for generation of clinical grade mRNA-transfected monocyte-derived dendritic cells for cancer vaccines. Scand. J. Immunol. 2003; 58:578-586.
53. Putz T., Gander H, Ramoner R et al. Generation of clinical-grade monocyte-derived dendritic cells using the ClinilVIACS system. Methods Mol. Med. 2005; 109:71-81.
54. Campbell J D M, Piechaczek C, Winkels G et al. Isolation and generation of clinical-grade dendritic cells using the CliniMACS system. Methods Mol. Med. 2005; 109:55-70.
55. Rieser C, Bock G, Klocker H, Bartsch G, Thurnher M. Prostaglandin E2 and tumor necrosis factor alpha cooperate to activate human dendritic cells: synergistic activation of interleukin 12 production. J Exp Med. 1997; 186:1603-1608.
56. Jonuleit H, Kuhn U, Muller G et al. Pro-inflammatory cytokines and prostaglandins induce maturation of potent immunostimulatory dendritic cells under fetal calf serum-free conditions. Eur J. Immunol. 1997; 27:3135-3142.
57. Coggin, J H, Jr. Embryonic antigens in malignancy and pregnancy: common denominators in immune regulation. Ciba Found. Symp. 1983; 96:28-54.
58. Rohrer J W, Culpepper C, Barsoum A L, Coggin J H, Jr. Characterization of RFM mouse T lymphocyte anti-oncofetal antigen immunity in apparent tumor-free, long-term survivors of sublethal x-irradiation by limiting dilution T lymphocyte cloning. J. Immunol. 1995; 154:2266-2280.
59. Rohrer J W, Coggin J H, Jr. CD8 T cell clones inhibit antitumor T cell function by secreting IL-10. J. Immunol. 1995; 155:5719-5727.
60. Therasse P, Arbuck S G, Eisenhauer E A et al. New guidelines to evaluate the response to treatment in solid tumors. European Organization for Research and Treatment of Cancer, National Cancer Institute of the United States, National Cancer Institute of Canada. J Nati Cancer Inst JID 7503089. 2000; 92:205-216.
61. Wolf C E, Meyer M, and Riggert, J. Leukapheresis for the extraction of monocytes and various lymphocyte subpopulations from peripheral blood: product quality and prediction of the yield using different harvest procedures. Vox Sanguinis 2005; 88:249-255.

62. Feuerstein B, Berger T G, Maczek C et al. A method for the production of cryopreserved aliquots of antigen-preloaded, mature dendritic cells ready for clinical use. J. Immunol. Methods 2000; 245:15-29.
63. Cao W, Lee S H, and Lu J. CD83 is preformed inside monocytes, macrophages, and dendritic cells, but is only stably expressed on activated dendritic cells. Biochem J. 2005; 385:85-93.
64. Zhou Le J and Tedder T F. CD14+ blood monocytes can differentiate into functionally mature CD83+ dendritic cells. Proc. Natl. Acad. Sci. (USA) 1996; 93:2588-2592.
65. Janeway C A, Travers P, Walport M et al. Immunobiology, 5th edition, Garland Publishing, New York, N.Y. 2001, p. 307.
66. Liu Y and Janeway Calif. Cells that present both specific ligand andco-stimulatory activity are the most efficient inducers of clonal expansion of normal CD4 T cells. Proc. Natl. Acad. Sci. (USA) 1992; 89:3845-3849.
67. Janeway Calif., Travers P, Walport M et al. Immunobiology, 5th edition, Garland Publishing, New York, N.Y. 2001, pp. 55, 75.
68. Mattes J, Hulett M, Xie W et al. Immunotherapy of cytotoxic T cell-resistant tumors by T helper 2 cells: an eotaxin and STAT6-dependent process. J. Exp. Med. 2003; 197:387-393.
69. Blum J L, Dieras V, Mucci Lo Russo P et al. Multicenter phase II study of capecitabine in taxane-pretreated metastatic breast carcinoma patients. Cancer 2001; 92:1759-1768.
70. Petit T, Benider A, Yovine A et al. Phase II study of an oxaliplatinlvinorelbine combination in patients with anthracycline- and taxanepre-treated metastatic breast cancer. Anticancer Drugs 2006; 17:337-343.
71. Esteva F J, Rivera E, Cristofanilli M et al. A phase II study of intravenous exatecan mesylate (DX-8951f) administered daily for 5 days every 3 weeks to patients with metastatic breast carcinoma. Cancer 2003; 98:900-907.
72. Keller A M, Mennel R G, Georgulias V A et al. Randomized phase III trial of pegylated liposomal doxorubicin versus vinorelbine or mitomycin C plus vinblastine in women with taxane-refractory advanced breast cancer. J. Clin. On_col. 2004; 22:3893-3901.
73. Rohrer J W, Barsoum A L, and Coggin J H, Jr. The development of a new universal tumor rejection antigen expressed on human and rodent cancers for vaccination, prevention of cancer, and antitumor therapy. Mod. Asp. Immunobiol. 2001; 191-195.

### INDUSTRIAL APPLICABILITY

The disclosed invention is useful in cancer therapy and treatment.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of producing an anti-cancer vaccine comprising autologous monocyte-derived oncofetal antigen-loaded (OFA)/immature laminin receptor protein (iLRP) mature dendritic cells, comprising the steps of:
(a) extracting and purifying CD14+ monocytes from a sample of blood collected from a cancer patient;
(b) cultivating the CD14+ monocytes with GM-CSF in a medium containing GM-CSF and IL-4 under conditions effective to induce monocytes to dendritic cell differentiation;
(c) contacting the cultivated immature dentritic cells of (b), in the same, replenished or different medium with an amount of OFA/iLRP, or a fragment thereof that selectively stimulates T cytotoxic lymphocytes, under conditions and in an amount of OFA/iLRP effective to allow uptake of the OFA/iLRP or fragment thereof by the dendritic cells;
(d) cultivating the OFA/iLRP-loaded dentritic cells of (c) with a dentritic cell maturation-inducing agent comprising a cocktail of cytokines under conditions and in amounts of the cytokines to allow the dendritic cells to mature; and
(e) harvesting the OFA/iLRP-loaded mature dendritic cells.

2. The method of claim 1, wherein the sample of peripheral blood is obtained from a breast cancer patient.

3. The method of claim 1, wherein the amount of GM-CSF ranges from about 2000 to about 3000 units per ml of medium.

4. The method of claim 1, wherein the amount of IL-4 ranges from about 800 to about 1200 units per ml of medium.

5. The method of claim 1, wherein said cultivating in (b) is conducted for about 4 to about 6 days.

6. The method of claim 1, wherein said contacting comprises cultivating the immature dendritic cells of (b) in medium comprising about 80 to about 120 nanograms per ml of the medium.

7. The method of claim 1, wherein in (c), said cocktail of cytokines comprises Il-1, Il-6 and TNF-α.

8. The method of claim 7, wherein said cocktail of cytokines further comprises PGE2.

9. The method of claim 8, wherein IL-1 is present in an amount of about 8 to about 12 nanograms per ml of the medium; IL-6 is present in an amount of about 800 to about 1200 units per ml of the medium; TNF-α is present in an amount of about 8 to about 12 nanograms per ml of the medium; and PGE2 is present in a concentration of about 0.8 to about 1.2 micromolar.

10. The method of claim 1, further comprising the step (f) of cryopreserving the harvested OFA/iLRP-loaded mature dendritic cells.

11. The method of claim 10, further comprising the step (g) of thawing and cultivating cryopreserved OFA/iLRP-loaded mature dendritic cells.

12. The method of claim 11, wherein in step (g) is conducted about 2 days prior to administration of the vaccine to the cancer patient.

13. The method of claim 12, which further comprises the step (h) of harvesting and resuspending the cultivated cryopreserved OFA/iLRP-loaded mature dendritic cells in a pharmaceutically acceptable carrier.

14. The method of claim 13, wherein the pharmaceutically acceptable carrier is lactated Ringer's solution containing autologous plasma.

15. The method of claim 14, wherein the autologous plasma is present in the carrier in a concentration of about 0.4% to about 0.6% (v/v).

16. A composition, comprising isolated monocyte-derived mature dendritic cells loaded with OFA/iLRP, or a fragment thereof that selectively stimulates T cytotoxic lymphocytes, and a carrier.

17. A vaccine composition, comprising an effective dosage amount of autologous, monocyte-derived mature dendritic cells loaded with OFA/iLRP or a fragment thereof that selectively stimulates T cytotoxic lymphocytes, and a pharmaceutically acceptable carrier.

18-21. (canceled)

22. A method of cancer treatment, comprising administering to a cancer patient a vaccine composition, comprising an effective dosage amount of autologous, monocyte-derived mature dendritic cells loaded with OFA/iLRP or a fragment thereof that selectively stimulates T cytotoxic lymphocytes, and a pharmaceutically acceptable carrier.

23-31. (canceled)

32. A method of monitoring the effect of cancer therapy, comprising:
(a) cultivating CD4 and CD8 T-lymphocytes isolated from a blood sample obtained from a cancer patient who has undergone treatment comprising administration of a vaccine composition comprising an effective dosage amount of autologous, monocyte-derived mature dendritic cells loaded with OFA/iLRP or a fragment thereof that selectively stimulates T cytotoxic lymphocytes, and a pharmaceutically acceptable carrier, wherein said cultivating is conducted in the presence of autologous, monocyte-derived mature dendritic cells loaded with OFA/iLRP isolated from the blood sample; and
(b) determining the frequency of gamma-interferon-secreting T lymphocytes and the frequency of Il-10-secreting T lymphocytes, each relative to a control, wherein an increased frequency of the gamma-interferon-secreting T lymphocytes relative to a frequency prior to the treatment is indicative that the treatment is effective.
